(19) United States
(12) Patent Application Publication
(10) Pub. No.: US 2022/0098162 A1
(43) Pub. Date: Mar. 31, 2022

(54) APHTHYLAMINE COMPOUND AND BIOLOGICALLY ACCEPTABLE SALT THEREOF, PREPARATION METHOD THEREFOR, AND APPLICATION THEREOF

(71) Applicant: Henan Radiomedical Science and Technology Co., Ltd, Kaifeng, Henan (CN)

(72) Inventors: Xuejun Xu, Kaifeng, Henan (CN); Yupo Yang, Kaifeng, Henan (CN); Zhengyan Yang, Kaifeng, Henan (CN)

(21) Appl. No.: 17/427,629

(22) PCT Filed: Aug. 27, 2020
(86) PCT No.: PCT/CN2020/111539

§ 371 Date: Jul. 31, 2021

(30) Foreign Application Priority Data
Aug. 27, 2019 (CN) 201910794306.1

(51) Int. Cl. C07D 295/088

(52) U.S. Cl. CPC C07D 295/088

(57) ABSTRACT

In view of the lack of anti-cancer targeted drugs in the prior art, the invention provides a naphthylamine compound and a biologically acceptable salt thereof, a preparation method thereof, and an application thereof. The naphthylamine compound and the biologically acceptable salt thereof provided by the invention can bind to protein sites related to tumor diseases in organisms through functional groups in the structure, and have hydrogen bonds and hydrophobic interactions with receptors, so as to achieve the purpose of inhibiting tumor cell proliferation.

### BACKGROUND OF THE INVENTION

### 1. Field of the Invention

The invention belongs to the technical field of tumor drug research and development, and specifically relates to a naphthylamine compound and a biologically acceptable salt thereof, a preparation method thereof, and an application thereof.

### 2. Description of the Related Art

Seeking new targets and potential drug lead compounds to make breakthroughs in specific tumor treatment fields is an urgent task for medical research and development researchers. The STAT3-JAK signal transduction pathway has a positive regulatory effect on the growth of tumor cells. In the past ten years, STAT3 protein has been favored as a biological target for the treatment of cancer. As of 2017, the US FDA has approved more than 30 lead compounds for STAT3 signaling pathway inhibitory anticancer drugs in clinical testing (Johnson D E, et al., Nature Reviews Clinical Oncology, 2018, 15(4):234). Anti-cancer targeted drugs based on STAT3 signal transduction have the characteristics of novel targets and broad anti-cancer spectrum, and recent clinical test results show that such drugs have huge development potential and broad market space in the future clinical treatment of tumors. Based on this, the invention explores new compounds that can be used to prepare anti-cancer targeted drugs based on STAT3 signal transduction.

### SUMMARY OF THE INVENTION

In view of the lack of anti-cancer targeted drugs in the prior art, the invention provides a naphthylamine compound and a biologically acceptable salt thereof, a preparation method thereof, and an application thereof. The naphthylamine compound and the biologically acceptable salt thereof provided by the invention can bind to protein sites related to tumor diseases in organisms through functional groups in the structure, and have hydrogen bonds and hydrophobic interactions with receptors, so as to achieve the purpose of inhibiting tumor cell proliferation.

The invention adopts the following technical solutions:

A naphthylamine compound, wherein the structural formula thereof is as shown in general formula I:

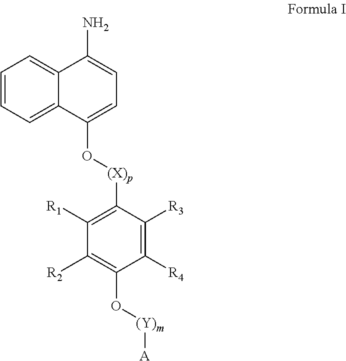

R₁, R₂, R₃, and R₄ are each independently selected from hydrogen, halogen, nitro, alkyl, cyano, and aryl;

p represents the number of X substituents, and P is 0 or 1;

X is —CH₂—, —(CH₂)₂—, —CO—, —CH₂—CO— or —(CH₂)₂—CO—;

m represents the number of Y substituents, and M is 0 or 1;

Y is —(CH₂)₂—, —(CH₂)₃—, —CO—, —CH₂—CO— or —(CH₂)₂—CO—;

A is

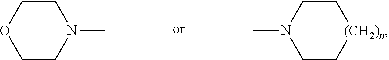

wherein n=0, 1, 2, 3.

The term “halogen” as used in the invention refers to fluorine, chlorine, bromine or iodine, and preferably, the halogen group is fluorine, chlorine or bromine. The naphthylamine compound is specifically a compound with the following structure:

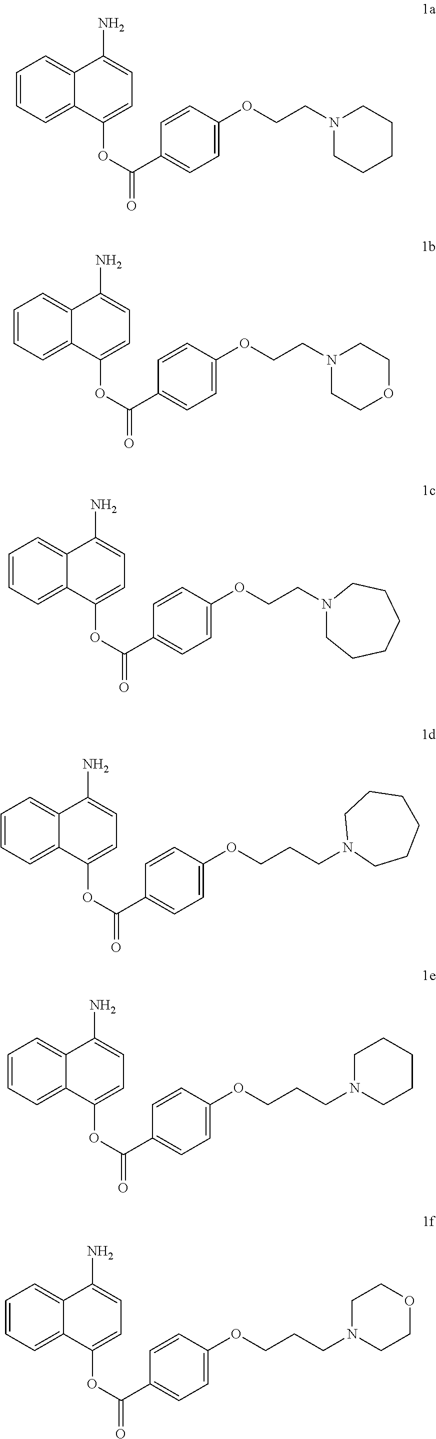 [SMILES: Nc1ccc(OC(=O)c2ccc(OCCN3CCCCC3)cc2)c2ccccc12 ; Nc1ccc(OC(=O)c2ccc(OCCN3CCOCC3)cc2)c2ccccc12 ; Nc1ccc(OC(=O)c2ccc(OCCN3CCCCCC3)cc2)c2ccccc12 ; Nc1ccc(OC(=O)c2ccc(OCCCN3CCCCCC3)cc2)c2ccccc12 ; Nc1ccc(OC(=O)c2ccc(OCCCN3CCCCC3)cc2)c2ccccc12 ; Nc1ccc(OC(=O)c2ccc(OCCCN3CCOCC3)cc2)c2ccccc12]

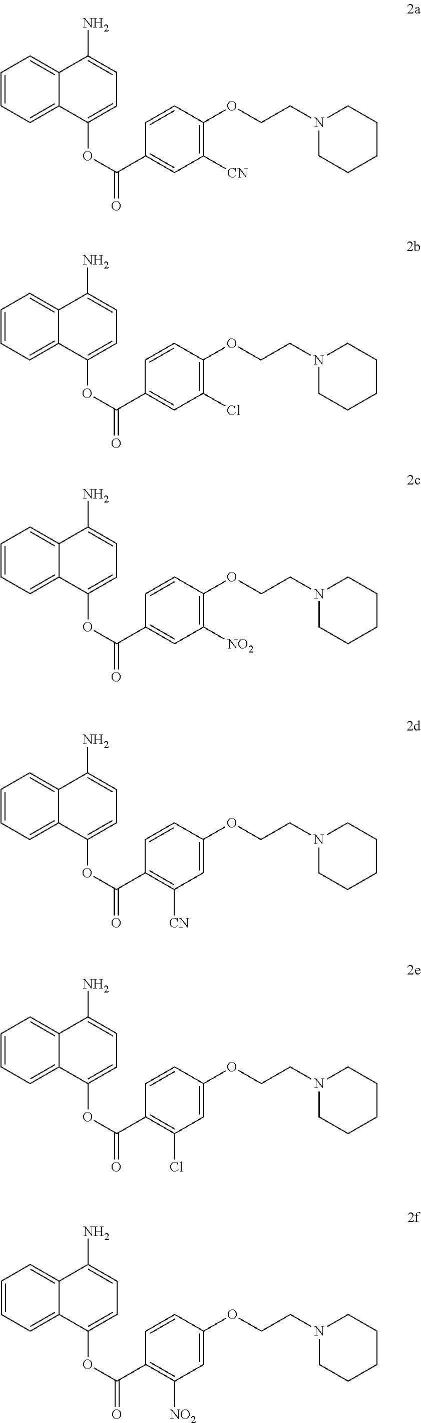 [SMILES: N#Cc1cc(C(=O)Oc2ccc(N)c3ccccc23)ccc1OCCN1CCCCC1 ; Nc1ccc(OC(=O)c2ccc(OCCN3CCCCC3)c(Cl)c2)c2ccccc12 ; Nc1ccc(OC(=O)c2ccc(OCCN3CCCCC3)c([N+](=O)[O-])c2)c2ccccc12 ; N#Cc1cc(OCCN2CCCCC2)ccc1C(=O)Oc1ccc(N)c2ccccc12 ; Nc1ccc(OC(=O)c2ccc(OCCN3CCCCC3)cc2Cl)c2ccccc12 ; Nc1ccc(OC(=O)c2ccc(OCCN3CCCCC3)cc2[N+](=O)[O-])c2ccccc12]

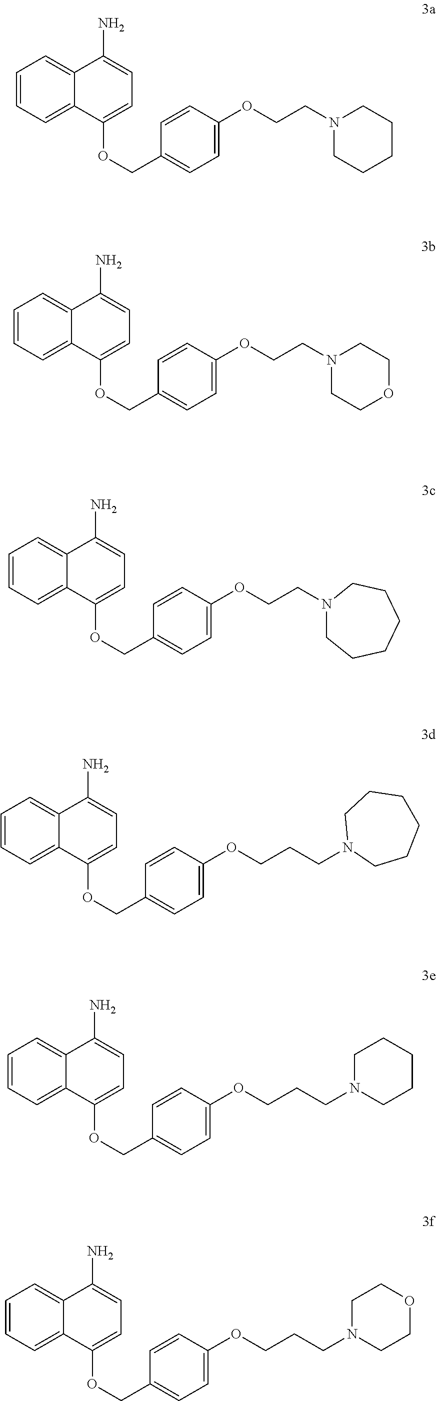 [SMILES: Nc1ccc(OCc2ccc(OCCN3CCCCC3)cc2)c2ccccc12 ; Nc1ccc(OCc2ccc(OCCN3CCOCC3)cc2)c2ccccc12 ; Nc1ccc(OCc2ccc(OCCN3CCCCCC3)cc2)c2ccccc12 ; Nc1ccc(OCc2ccc(OCCCN3CCCCCC3)cc2)c2ccccc12 ; Nc1ccc(OCc2ccc(OCCCN3CCCCC3)cc2)c2ccccc12 ; Nc1ccc(OCc2ccc(OCCCN3CCOCC3)cc2)c2ccccc12]

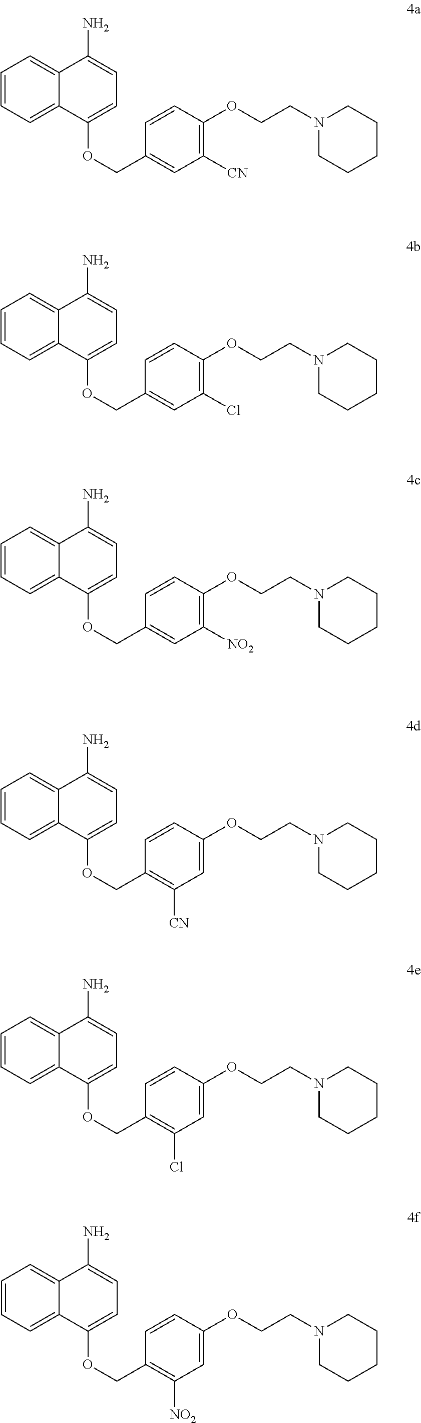 [SMILES: N#Cc1cc(COc2ccc(N)c3ccccc23)ccc1OCCN1CCCCC1 ; Nc1ccc(OCc2ccc(OCCN3CCCCC3)c(Cl)c2)c2ccccc12 ; Nc1ccc(OCc2ccc(OCCN3CCCCC3)c([N+](=O)[O-])c2)c2ccccc12 ; N#Cc1cc(OCCN2CCCCC2)ccc1COc1ccc(N)c2ccccc12 ; Nc1ccc(OCc2ccc(OCCN3CCCCC3)cc2Cl)c2ccccc12 ; Nc1ccc(OCc2ccc(OCCN3CCCCC3)cc2[N+](=O)[O-])c2ccccc12]

A biologically acceptable salt formed by the naphthylamine compound with at least one of acetic acid, dihydroacetic acid, benzoic acid, citric acid, sorbic acid, propionic acid, oxalic acid, fumaric acid, maleic acid, hydrochloric acid, malic acid, phosphoric acid, sulfurous acid, sulfuric acid, vanillic acid, tartaric acid, ascorbic acid, boric acid, lactic acid and ethylenediaminetetraacetic acid.

A preparation method of the naphthylamine compound, comprising the following steps:

(1) dissolving

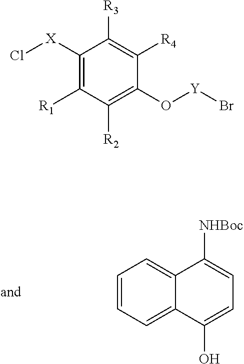

with a molar ratio of 1:1 in an organic solvent, and adding an alkali; after the reaction is detected by TLC,

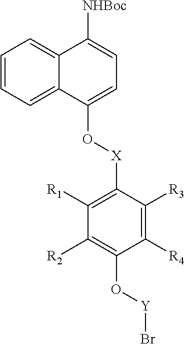

is obtained by post-processing;

(2) then

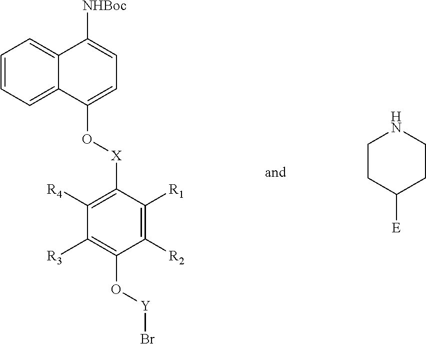

with a molar ratio of 1:3 are subjected to a nucleophilic substitution reaction to generate

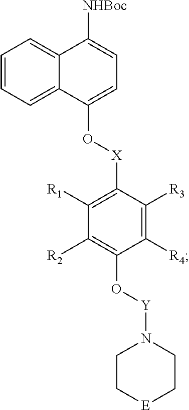

wherein E is —CH₂—, —O— or —(CH₂)₂—.

Further, the

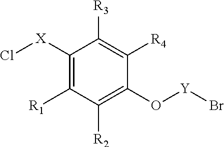

is prepared by the following method:

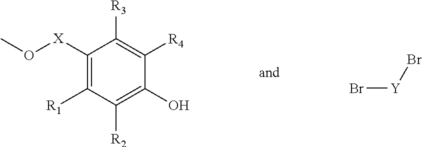

with a molar ratio of 1:4 are subjected to a nucleophilic substitution reaction to obtain

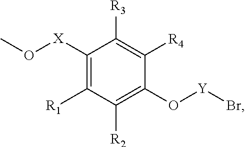

and then

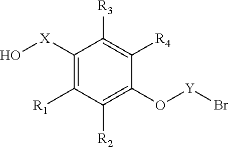

is converted to

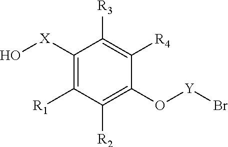

through the reaction, and then is subjected to a halogenating reaction with a chlorinating agent to obtain the

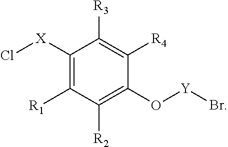

Further, the

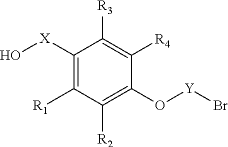

comprises

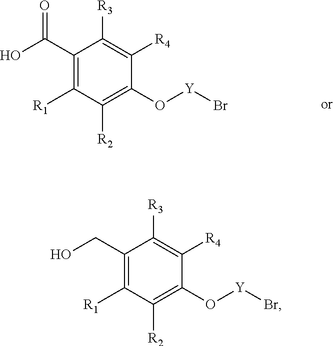

and the specific preparation method of

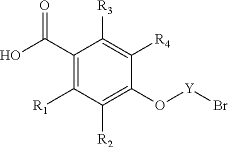

is as follows:

dissolving the

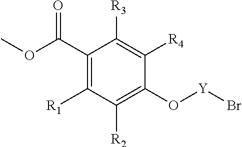

in a mixed solvent of tetrahydrofuran and water, then adding lithium hydroxide, and reacting at 20-50° C.; after the reaction is detected by TLC, removing the tetrahydrofuran by rotary evaporation; adjusting the pH value of the residue to 1-3 with hydrochloric acid, and the solid obtained by precipitation is

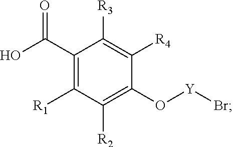

the specific preparation method of

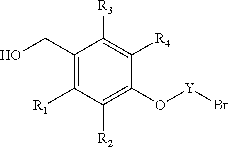

is as follows:

dissolving

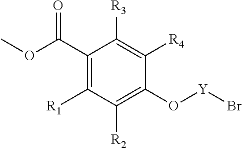

in tetrahydrofuran, adding lithium aluminum tetrahydrogen, and reacting at room temperature; after the reaction is detected by TLC, pouring the reaction solution into water; adjusting the pH value to 1-3 with hydrochloric acid, extracting with ethyl acetate, collecting the organic phase, filtering and performing rotary evaporation to obtain it.

Further, when the structural formula of the compound is 1a to 2f, Y is —(CH₂)₂— or —(CH₂)₃— at the moment, and the specific preparation method thereof is as follows:

(1) dissolving

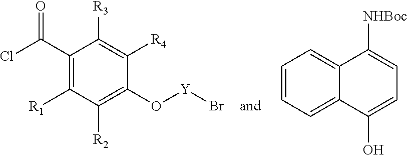

in tetrahydrofuran, adding triethylamine, and reacting at room temperature; after the reaction is detected by TLC,

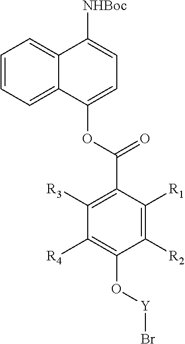

is obtained by post-processing;

wherein the molar ratio of

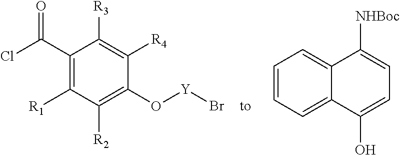

to triethylamine is 1:1:2;

(2) then dissolving

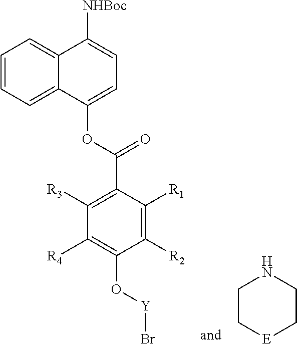

in tetrahydrofuran, adding potassium iodide, and after the reflux reaction is completed, it is obtained by post-processing;

wherein the molar ratio of

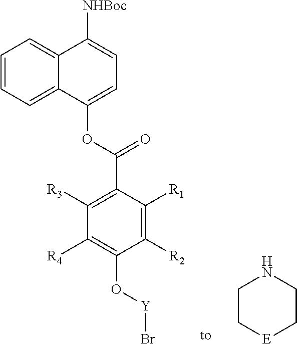

to potassium iodide is 1:3:0.1.

Further, when the structural formula of the compound is 3a to 4f, Y is —(CH₂)₂— or —(CH₂)₃— at the moment, and the specific preparation method thereof is as follows:

(1) dissolving

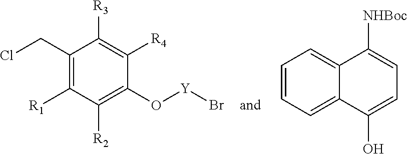

in acetonitrile, adding potassium carbonate, and reacting at 60-80° C.; after the reaction is detected by TLC,

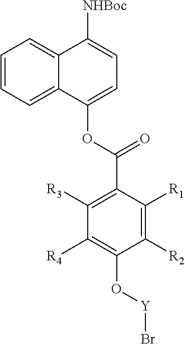

is obtained by post-processing;

wherein the molar ratio of

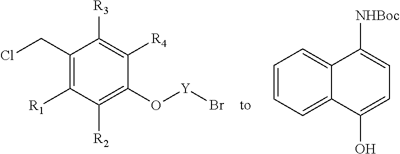

to potassium carbonate is 1:1:1.2;

(2) then dissolving

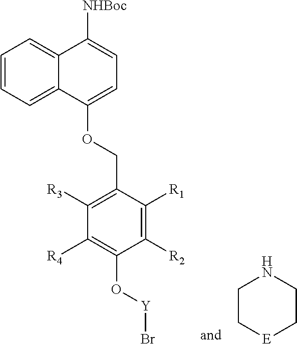

in tetrahydrofuran, adding potassium iodide, and after the reflux reaction is completed, it is obtained by post-processing;

wherein the molar ratio of

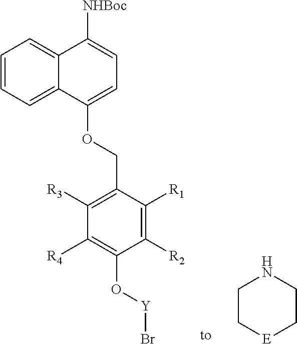

to potassium iodide is 1:3:0.1.

The biologically acceptable salt of the naphthylamine compound is prepared by the following method: dissolving the naphthylamine compound in the methanol solution of the corresponding acid, and reacting at room temperature; after the reaction is detected by TLC, it is obtained by post-processing.

According to the structural differences of the naphthylamine compounds of the above formula I, the invention provides two preparation methods at the same time, as follows:

The naphthylamine compounds with structural formulas 1a to 2f can be synthesized by the route shown in Scheme 1. The raw material undergoes a two-step nucleophilic substitution reaction to form an etherified intermediate, which is then hydrolyzed by a strong base solution to form the corresponding carboxylic acid. After the carboxylic acid is purified, the acylation reaction is carried out to generate the corresponding acid chloride, and then the target compound precursor with a protective group is synthesized in a basic environment, and finally the target compound is obtained by deprotection under acidic conditions.

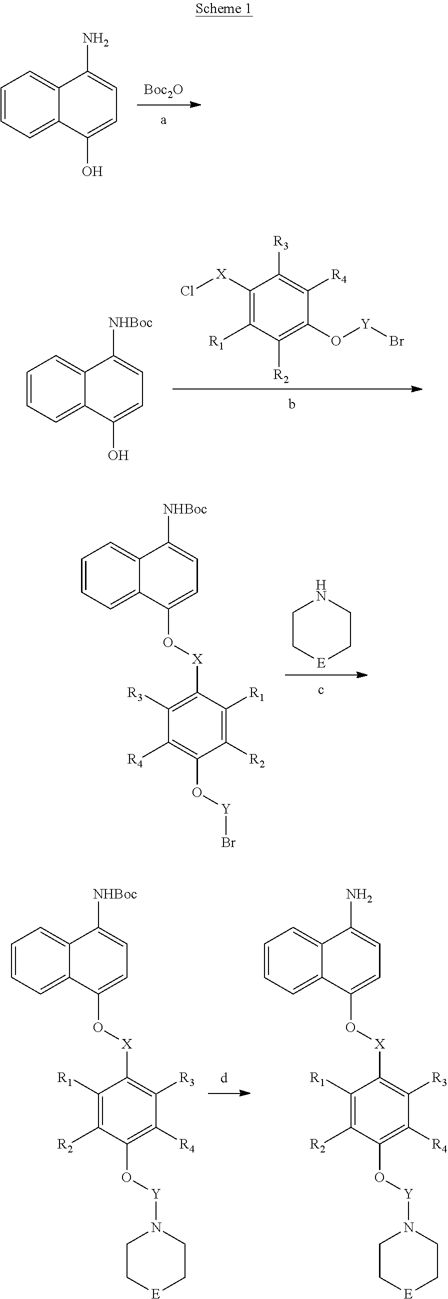

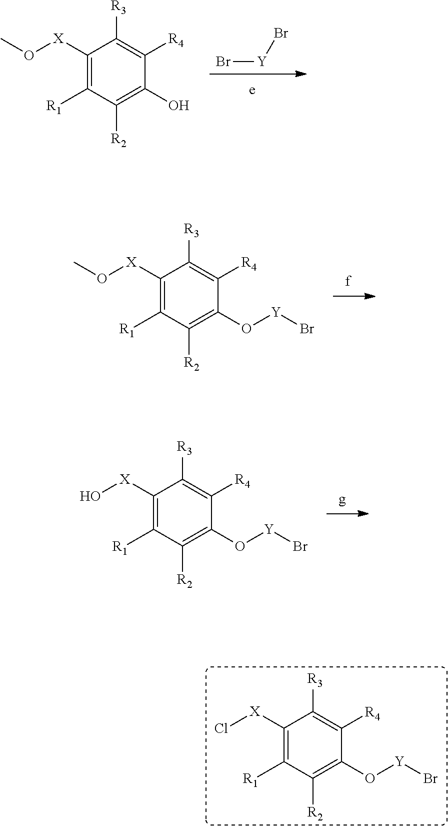

For the naphthylamine compounds shown in structural formulas 1a to 2f, the above substituents are specifically:

X=—CO—, Y=—(CH₂)₂—, E=—CH₂—, R₁=H, R₂=H, R₃=H, R₄=H; 1a:

X=—CO—, Y=—(CH₂)₂—, E=—O—, R₁=H, R₂=H, R₃=H, R₄=H; 1b:

X=—CO—, Y=—(CH₂)₂—, E=—(CH₂)₂—, R₁=H, R₂=H, R₃=H, R₄=H; 1c:

X=—CO—, Y=—(CH₂)₃—, E=—(CH₂)₂—, R₁=H, R₂=H, R₃=H, R₄=H; 1d:

X=—CO—, Y=—(CH₂)₃—, E=—CH₂—, R₁=H, R₂=H, R₃=H, R₄=H; 1e:

X=—CO—, Y=—(CH₂)₃—, E=—O—, R₁=H, R₂=H, R₃=H, R₄=H; 1f:

X=—CO—, Y=—(CH₂)₂—, E=—CH₂—, R₁=H, R₂=CN, R₃=H, R₄=H; 2a:

X=—CO—, Y=—(CH₂)₂—, E=—O—, R₁=H, R₂=Cl, R₃=H, R₄=H; 2b:

X=—CO—, Y=—(CH₂)₂—, E=—(CH₂)₂—, R₁=H, R₂=NO₂, R₃=H, R₄=H; 2c:

X=—CO—, Y=—(CH₂)₃—, E=—(CH₂)₂—, R₁=CN, R₂=H, R₃=H, R₄=H; 2d:

X=—CO—, Y=—(CH₂)₃—, E=—CH₂—, R₁=C1, R₂=H, R₃=H, R₄=H; 2e:

X=—CO—, Y=—(CH₂)₃—, E=—O—, R₁=NO₂, R₂=H, R₃=H, R₄=H. 2f:

The specific groups for X, Y, and E include the above 1a, 1b, 1c, 1d, 1e, 1f, 2a, 2b, 2c, 2d, 2e, and 2f corresponding groups, but are not limited to these groups/compounds, and can also be other compounds that can be easily understood by those skilled in the art to use this Scheme 1 for synthesis. The compounds in Scheme 2 hereinafter have the same definitions for X, Y, and E as in the above cases, including but not limited to these specific compounds.

The naphthylamine compounds with structural formulas 3a to 4f can be synthesized by the route shown in Scheme 2. The raw material is reduced by tetrahydroaluminum lithium, and thionyl chloride is reacted to obtain a chlorinated hydrocarbon intermediate. After a two-step nucleophilic substitution reaction, a protected target precursor is obtained. Finally, the target compound is deprotected under acidic conditions.

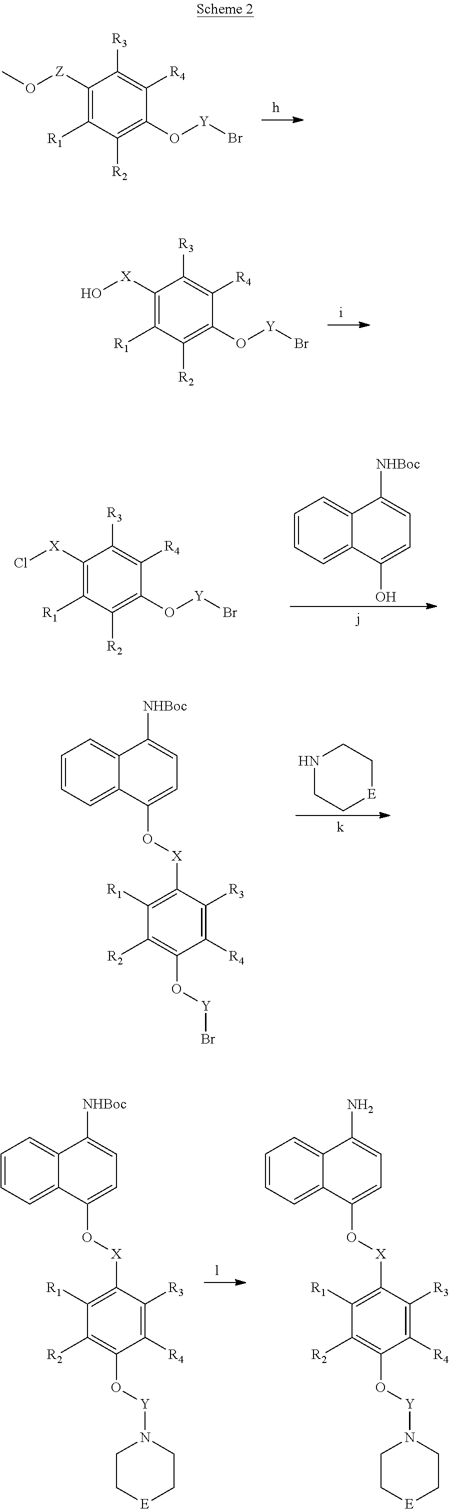

For the naphthylamine compounds shown in structural formulas 3a to 4f, the above substituents are specifically:

X=—CH₂—, Y=—(CH₂)₂—, Z=—CH₂—, E=—CH₂—, R₁=H, R₂=H, R₃=H, R₄=H; 3a:

X=—CH₂—, Y=—(CH₂)₂—, Z=—CH₂—, E=—O—, R₁=H, R₂=H, R₃=H, R₄=H; 3b:

X=—CH₂—, Y=—(CH₂)₂—, Z=—CH₂—, E=—(CH₂)₂—, R₁=H, R₂=H, R₃=H, R₄=H; 3c:

X=—CH₂—, Y=—(CH₂)₃—, Z=—CH₂—, E=—(CH₂)₂—, R₁=H, R₂=H, R₃=H, R₄=H; 3d:

X=—CH₂—, Y=—(CH₂)₃—, Z=—CH₂—, E=—CH₂—, R₁=H, R₂=H, R₃=H, R₄=H; 3e:

X=—CH₂—, Y=—(CH₂)₃—, Z=—CH₂—, E=—O—, R₁=H, R₂=H, R₃=H, R₄=H; 3f:

X=—CH₂—, Y=—(CH₂)₂—, Z=—CH₂—, E=—CH₂—, R₁=H, R₂=CN, R₃=H, R₄=H; 4a:

X=—CH₂—, Y=—(CH₂)₂—, Z=—CH₂—, E=—O—, R₁=H, R₂=Cl, R₃=H, R₄=H; 4b:

X=—CH₂—, Y=—(CH₂)₂—, Z=—CH₂—, E=—(CH₂)₂—, R₁=H, R₂=NO₂, R₃=H, R₄=H; 4c:

X=—CH₂—, Y=—(CH₂)₃—, Z=—CH₂—, E=—(CH₂)₂—, R₁=CN, R₂=H, R₃=H, R₄=H; 4d:

X=—CH₂—, Y=—(CH₂)₃—, Z=—CH₂—, E=—CH₂—, R₁=Cl, R₂=H, R₃=H, R₄=H; 4e:

X=—CH₂—, Y=—(CH₂)₃—, Z=—CH₂—, E=—O—, R₁=NO₂, R₂=H, R₃=H, R₄=H. 4f:

The purpose of the invention is to find new compounds with high inhibitory effect on STAT3 and lower toxicity.

The invention further relates to the application of the naphthylamine compound, the pharmaceutically acceptable salt thereof, the solvent compound of the derivative, or the solvent compound of the salt in the preparation of a medicament for the treatment or adjuvant treatment and/or prevention of tumors in mammals, which is mainly used in drugs for tumors mediated by STAT3 or tumor cell proliferation and migration driven by STAT3, and can also be drugs for diseases related to STAT3 cell signal transduction. Specifically, the mammals are human beings.

One aspect of the invention relates to the application of the above novel naphthylamine compound with the structure of formula I, the pharmaceutically acceptable salt thereof, the solvent compound of the derivative, or the solvate of the salt in the preparation of a medicament for treating and/or preventing diseases related to STAT3 cell signaling in mammals. Specifically, the mammals are human beings.

According to the invention, it is entirely expected that the compounds of the invention can be used to treat tumors caused by abnormally active STAT3 signal transduction or high protein expression. STAT3-related tumors include lung cancer, breast cancer, colorectal cancer, leukemia, head and neck cancer, prostate cancer and all other cancers.

The advantageous effects of the invention are:

The invention discloses a new type of naphthylamine compound and a salt preparation method, and the application of such compound and the salt form thereof as active ingredients in cell growth regulation mechanisms and cancer treatment. The naphthylamine compound and the salt form thereof, due to its unique structural characteristics, can bind to protein sites related to tumor diseases in organisms through functional groups in the structure, and have hydrogen bonds and hydrophobic interactions with receptors, so as to achieve the purpose of inhibiting tumor cell proliferation. For example, naphthylamine compounds such as SMY001 and SMY002 belong to STAT3 inhibitors, and the mechanism of such compounds to inhibit STAT3 activation is clear and the effect of inhibiting tumor cell growth is significant. The biological activity test shows that the compound of the invention has a significant inhibitory effect on the signal transduction pathway of STAT3 cells in tumor cells; specifically, it has a significant inhibitory effect on the activation of phosphorylated STAT3 protein and the expression of downstream genes, and has a significant antagonistic effect on the growth and reproduction of various cancer cells such as lung cancer, breast cancer and colon cancer. This indicates that such compounds have potentially important significance and broad application prospects for tumor mechanism research and cancer clinical treatment.

### BRIEF DESCRIPTION OF THE DRAWINGS

FIG. 1 is an MTT experiment of compounds SMY001 (1a) and SMY002 (3a) inducing apoptosis of breast cancer cells MDA-MB-231 cells; the results of the MTT cell experiment in the figure are characterized by IC50 (μmol/L) value.

FIG. 2 is an MTT experiment of compounds SMY001 (1a) and SMY002 (3a) inducing apoptosis of breast cancer cells MCF-7 cells; the results of the MTT cell experiment in the figure are characterized by IC50 (μmol/L) value.

FIG. 3 is an MTT experiment of compounds SMY001 (1a) and SMY002 (3a) inducing apoptosis of breast cancer cells HCT-116 cells; the results of the MTT cell experiment in the figure are characterized by IC50 (μmol/L) value.

FIG. 4 is an MTT experiment of compounds SMY001 (1a) and SMY002 (3a) inducing apoptosis of breast cancer cells PC9-AR cells; the results of the MTT cell experiment in the figure are characterized by IC50 (μmol/L) value.

FIG. 5 is an MTT experiment of compounds SMY001 (1a) and SMY002 (3a) inducing apoptosis of breast cancer cells PC9-GR cells; the results of the MTT cell experiment in the figure are characterized by IC50 (μmol/L) value.

FIG. 6 is an MTT experiment of compounds SMY001 (1a) and SMY002 (3a) inducing apoptosis of breast cancer cells PC9 cells; the results of the MTT cell experiment in the figure are characterized by IC50 (μmol/L) value.

FIG. 7 is the result of Westernblot of the compound SMY002 (3a).

FIG. 8 is the results of the docking experiment.

### DETAILED DESCRIPTION OF THE PREFERRED EMBODIMENTS

In order to make the technical objectives, technical solutions and advantageous effects of the invention clearer, the technical solutions of the invention will be further described hereinafter with reference to the drawings and specific embodiments.

In the method for synthesizing the compound of formula I of the invention, the various raw materials used in the reaction can be prepared by those skilled in the art based on existing knowledge, or can be prepared by methods known in the literature, or can be commercially available. The intermediates, raw materials, reagents, reaction conditions, etc. used in the above reaction schemes can be appropriately changed according to the knowledge of those skilled in the art.

In the invention, unless otherwise specified, wherein: (i) the temperature is expressed in degrees Celsius (° C.), and the operation is carried out in a room temperature environment; more specifically, the room temperature refers to 20° C.-30° C.; (ii) the organic solvent is dried by a common drying method, and the solvent is evaporated under reduced pressure with a rotary evaporator, and the bath temperature is not higher than 50° C.; the developing agent and eluent are in volume ratio; (iii) the reaction process is followed by thin layer chromatography (TLC); (iv) the final product has satisfactory proton nuclear magnetic resonance (1H-NMR).

### Embodiment 1: Synthesis of Compounds 1a-2f

With Reference to Scheme 1

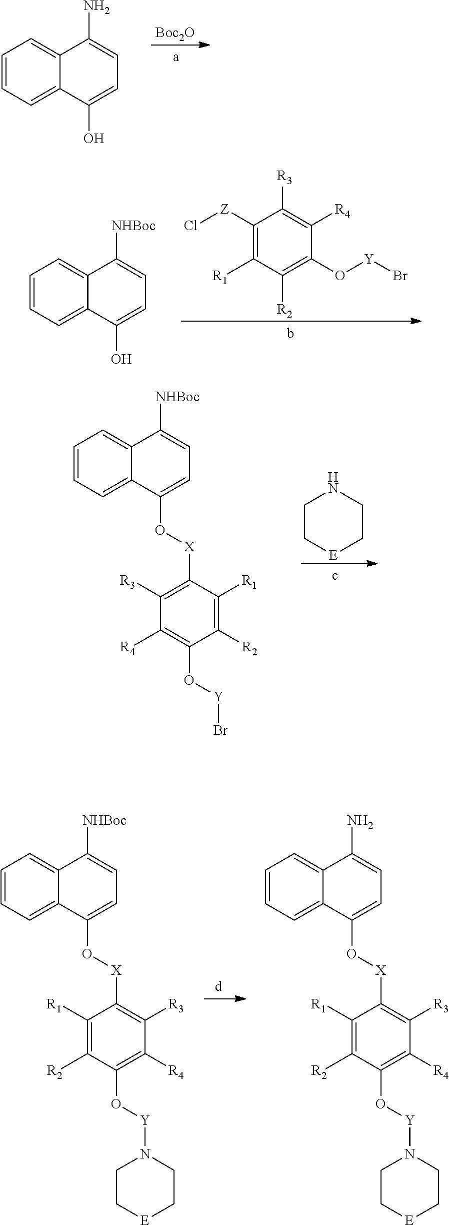

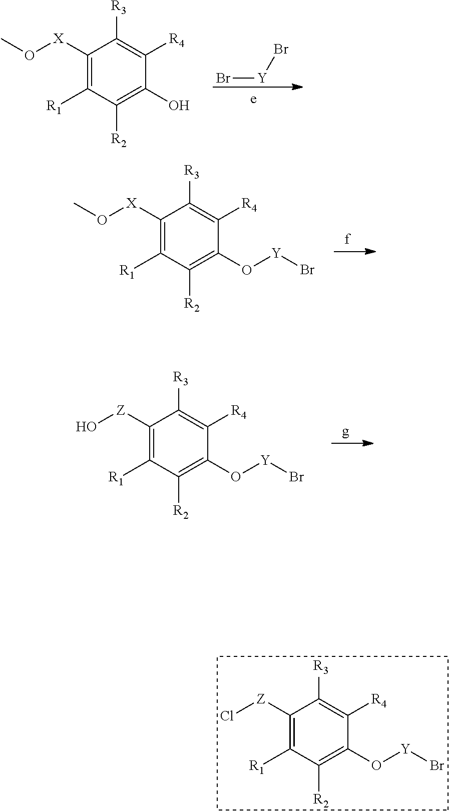

X=—CO—, Y=—(CH₂)₂—, E=—CH₂—, R₁=H, R₂=H, R₃=H, R₄=H; 1a:

X=—CO—, Y=—(CH₂)₂—, E=—O—, R₁=H, R₂=H, R₃=H, R₄=H; 1b:

X=—CO—, Y=—(CH₂)₂—, E=—(CH₂)₂—, R₁=H, R₂=H, R₃=H, R₄=H; 1c:

X=—CO—, Y=—(CH₂)₃—, E=—(CH₂)₂—, R₁=H, R₂=H, R₃=H, R₄=H; 1d:

X=—CO—, Y=—(CH₂)₃—, E=—CH₂—, R₁=H, R₂=H, R₃=H, R₄=H; 1e:

X=—CO—, Y=—(CH₂)₃—, E=—O—, R₁=H, R₂=H, R₃=H, R₄=H; 1f:

X=—CO—, Y=—(CH₂)₂—, E=—CH₂—, R₁=H, R₂=CN, R₃=H, R₄=H; 2a:

X=—CO—, Y=—(CH₂)₂—, E=—O—, R₁=H, R₂=Cl, R₃=H, R₄=H; 2b:

X=—CO—, Y=—(CH₂)₂—, E=—(CH₂)₂—, R₁=H, R₂=NO₂, R₃=H, R₄=H; 2c:

X=—CO—, Y=—(CH₂)₃—, E=—(CH₂)₂—, R₁=CN, R₂=H, R₃=H, R₄=H; 2d:

X=—CO—, Y=—(CH₂)₃—, E=—CH₂—, R₁=C1, R₂=H, R₃=H, R₄=H; 2e:

X=—CO—, Y=—(CH₂)₃—, E=—O—, R₁=NO₂, R₂=H, R₃=H, R₄=H. 2f:

The specific synthesis method is as follows, taking the compound 1a as an example, whose structural formula is below:

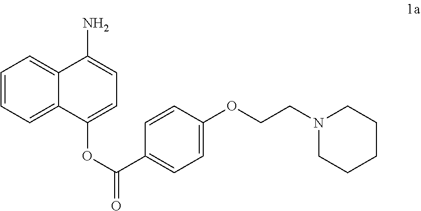 [SMILES: Nc1ccc(OC(=O)c2ccc(OCCN3CCCCC3)cc2)c2ccccc12]

Compound 1a is named 4-(2-(piperidin-1-yl) ethoxy) benzoic acid-4-amino-1-naphthyl ester dihydrochloride, and the synthetic route thereof is shown below:

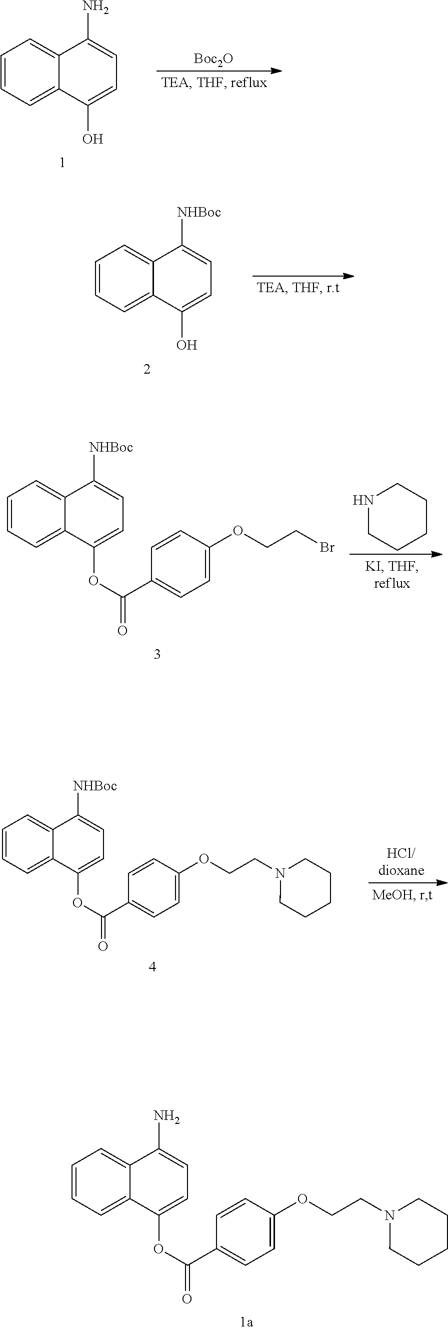

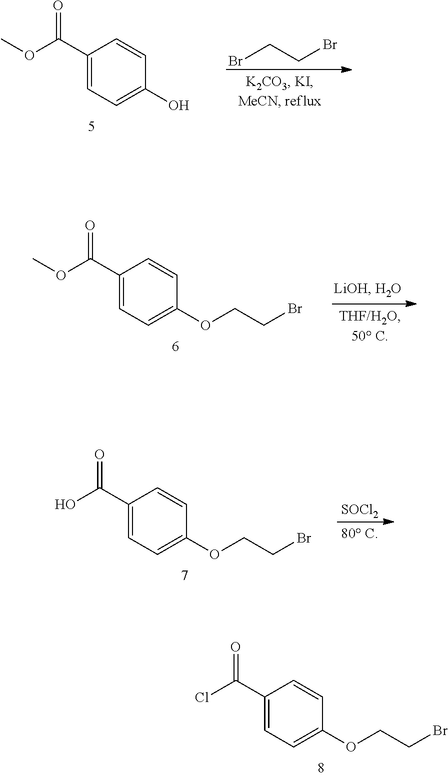

### Step 1. 1-tert-Butoxycarbonylamino-4-hydroxy-naphthalene (2)

Dissolving 4-Amino-1-naphthol (1) (2.00 g, 12.6 mmol, 1.0 eq), Boc₂O (di-tert-butyl dicarbonate, 3.29 g, 15.1 mmol, 1.2 eq), 4-dimethylaminopyridine (153 mg, 1.26 mmol, 0.1 eq), and triethylamine (2.80 g, 27.6 mmol, 2.20 eq) in tetrahydrofuran (20 mL), and heating up to 78° C. to react for 2 hours. TLC (petroleum ether:ethyl acetate=1:1, R_{f}/compound 1=0.30, R_{f}/compound 2=0.75) shows that the reaction of the raw materials is completed. Cooling the reaction solution to room temperature, pouring into water (50 mL), extracting with ethyl acetate (50 mL*3), combining the organic phases and drying with anhydrous sodium sulfate, and then spin-drying to obtain a crude product. Purifying crude product by column chromatography (petroleum ether/ethyl acetate=5:1-3:1) to obtain 2.60 g of 1-tert-butoxycarbonylamino-4-hydroxy-naphthalene (2), a brown oily liquid, with a yield of 79.8%.

¹H NMR (CDCl₃, 300 MHz) δ: 7.90 (d, J=6.0 Hz, 1H), 7.78 (d, J=6.0 Hz, 1H), 7.54-7.45 (m, 2H), 7.11 (d, J=9.0 Hz, 1H), 6.67 (d, J=9.0 Hz, 1H), 3.80 (brs, 2H), 1.59 (s, 9H).

### Step 2. 4-(2-Bromoethoxy) benzoic acid-4-(tert-butoxycarbonyl) amino-1-naphthyl Ester (3)

Dissolving 1-tert-Butoxycarbonylamino-4-hydroxy-naphthalene (2) (148 mg, 0.57 mmol, 1.0 eq), 4-(2-bromoethoxy) benzoyl chloride (8) (150 mg, 0.57 mmol, 1.0 eq), and triethylamine (115 mg, 1.12 mmol, 2.0 eq) in tetrahydrofuran (5 mL) and reacting at room temperature for 12 hours. TLC (petroleum ether:ethyl acetate=2:1, R_{f}/compound 2=0.60, R_{f}/compound 3=0.75) shows that the raw material is consumed. Pouring the reaction solution into 20 mL water, extracting 3 times with ethyl acetate (20 mL*3), combining the organic phases, drying with anhydrous sodium sulfate and spin-drying, purifying by column chromatography (petroleum ether:ethyl acetate=10:1-3:1) to obtain 154 mg of 4-(2-bromoethoxy) benzoic acid-4-(tert-butoxycarbonyl) amino-1-naphthyl ester (3), a pale yellow solid, with a yield of 55.6%.

¹H NMR (DMSO-d⁶, 300 MHz) δ: 10.32 (s, 1H), 8.85 (d, J=9.0 Hz, 2H), 8.02 (d, J=8.0 Hz, 1H), 7.86 (d, J=8.0 Hz, 1H), 7.67-7.58 (m, 3H), 7.42 (d, J=8.0 Hz, 1H), 7.13 (d, J=9.0 Hz, 2H), 4.45 (t, J=8.0 Hz, 2H), 3.86 (t, J=8.0 Hz, 2H), 1.55 (s, 9H).

### Step 3. 4-(2-(piperidin-1-yl) ethoxy) benzoic acid-4-(tert-butoxycarbonyl) amino-1-naphthyl Ester (4)

Dissolving 4-(2-Bromoethoxy) benzoic acid-4-(tert-butoxycarbonyl) amino-1-naphthyl ester (3) (154 mg, 0.32 mmol, 1.0 eq), piperidine (80.9 mg, 0.94 mmol, 3.0 eq) and potassium iodide (5.26 mg, 0.032 mmol, 0.1 eq) in 5 mL of tetrahydrofuran, and heating up to 78° C. to react for 12 hours. TLC (dichloromethane:methanol=10:1, R_{f}/compound 3=0.95, R_{f}/compound 4=0.30) shows that the reaction of the raw materials is completed. Cooling the reaction solution to room temperature, pouring into 20 mL of water, extracting three times with 60 mL of ethyl acetate (20 mL*3), combining the organic phases, drying over anhydrous sodium sulfate, and spin-drying. Purifying the crude product by column chromatography (dichloromethane:methanol=100:1-20:1) to obtain 60 mg of 4-(2-(piperidin-1-yl) ethoxy) benzoic acid-4-(tert-Butoxycarbonyl) amino-1-naphthyl ester (4) as a pale yellow solid, with a yield of 38.8%.

### Step 4. 4-(2-(piperidin-1-yl) ethoxy) benzoic acid-4-amino-1-naphthyl Ester Dihydrochloride (Hydrochloride of Compound 1a)

Dissolving 4-(2-(piperidin-1-yl) ethoxy) benzoic acid-4-(tert-Butoxycarbonyl) amino-1-naphthyl ester (4) (60.0 mg, 0.122 mmol, 1.0 eq) in 2 mL of methanol, slowly dropping in 2 mL of HCl/methanol solution (6 mol/L), and reacting at room temperature for 12 hours. TLC (dichloromethane:methanol=10:1, R_{f}/compound 4=0.30, R_{f}/1a=0.15) shows that the reaction of the raw materials is completed and a new point is formed. Spin-drying the reaction solution, and stripping with anhydrous toluene three times to obtain 45 mg of 4-(2-(piperidin-1-yl) ethoxy) 4-amino-1-naphthyl benzoate dihydrochloride (1a), a pale yellow solid, with a yield of 80%.

¹H NMR (DMSO-d⁶, 300 MHz) δ: 10.16 (brs, 1H), 10.0 (brs, 1H), 8.17 (d, J=9.0 Hz, 1H), 8.04 (d, J=9.0 Hz, 2H), 7.81 (d, J=9.0 Hz, 1H), 7.48 (m, 2H), 7.29 (d, J=8.0 Hz, 1H), 7.07 (d, J=9.0 Hz, 2H), 6.88 (d, J=8.0 Hz, 1H), 4.18 (t, J=8.0 Hz, 2H), 2.73 (t, J=8.0 Hz, 2H), 2.53 (m, 4H), 1.55-1.53 (m, 4H), 1.41 (m, 2H).

Wherein in step 2, the synthesis method of 4-(2-bromoethoxy) benzoyl chloride (8) used is as follows:

### (i) Methyl 4-(2-bromoethoxy) benzoate (6)

Dissolving methyl 4-hydroxybenzoate (5) (1.00 g, 6.57 mmol, 1.0 eq), 1, 2-dibromoethane (4.94 g, 26.3 mmol, 4.0 eq), potassium carbonate (1.18 g, 8.54 mmol, 1.30 eq) and potassium iodide (109 mg, 0.66 mol, 0.10 eq) in 15 mL of acetonitrile and heating to 80° C. for 12 hours. TLC (petroleum ether:ethyl acetate=3:1, R_{f}/compound 5=0.40, R_{f}/compound 6=0.75) shows that most of the raw materials are consumed, a small amount of raw materials remain and new spots are generated. Cooling the reaction solution to room temperature, pouring into 50 mL of water, extracting with 150 mL of ethyl acetate (50 mL*3), combining the organic phases, and drying with anhydrous sodium sulfate and then spin-drying.

¹H NMR (CDCl₃, 300 MHz) δ: 7.93 (d, J=6.0 Hz, 2H), 6.85 (d, J=6.0 Hz, 2H), 4.30 (t, J=6.0 Hz, 2H), 3.74 (s, 3H), 3.58 (t, J=6.0 Hz, 2H).

### (ii) 4-(2-Bromoethoxy) benzoic Acid (7)

Dissolving 4-(2-bromoethoxy) methyl benzoate (6) (500 mg, 1.93 mmol, 1.0 eq) in 10 mL of tetrahydrofuran and 2 mL of water, then adding lithium hydroxide monohydrate (162 mg, 3.86 mmol, 2.0 eq), and heating up the reaction solution to 50° C. for 2 hours. TLC (petroleum ether:ethyl acetate=3:1, R_{f}/compound 6=0.75, R_{f}/compound 7=0.05) shows that the reaction of the raw materials is completed and new spots are generated. Revolving most of the tetrahydrofuran under reduced pressure, adjusting the remaining reaction liquid to pH=1-3 with dilute hydrochloric acid. Solids precipitate out. Filtering and collecting the filter cake and stripping with toluene three times to obtain 420 mg of 4-(2-bromoethoxy) benzoic acid (7), a white solid, with a yield of 88.8%.

¹HNMR (DMSO-d⁶, 300 MHz) δ: 12.65 (brs, 1H), 7.91-7.87 (m, 2H), 7.07-7.01 (m, 2H), 4.48-4.16 (m, 2H), 3.85-3.66 (m, 2H).

### (iii) 4-(2-Bromoethoxy) benzoyl Chloride (8)

Dissolving 4-(2-Bromoethoxy) benzoic acid (7) (140 mg, 0.57 mmol, 1.0 eq) in thionyl chloride (3 mL), and heating up to 80° C. to react for 2 hours. Spin-drying the reaction solution and stripping with dichloromethane three times (20 mL*3) to obtain 150 mg of 4-(2-bromoethoxy) benzoyl chloride (8), which is used directly in the next reaction without purification.

Refer to Embodiment 1 for the synthesis method of the hydrochloride salt of compounds 1b-2f, the difference is: in the synthesis process of compound 1b and 1c, in step 3, morpholine and cycloheximide (ie homopiperidine) are used to replace piperidine, respectively, and the remaining steps are the same as in Embodiment 1; in the synthesis process of compounds 1d, 1e, and 1f, in step (i), 1,3-dibromopropane is used to replace 1,2-dibromopropane; in the synthesis process of compounds 1d and 1f, in step 3, morpholine and cycloheximine (i.e., homopiperidine) are used to replace piperidine, respectively, and the remaining steps are the same as in Embodiment 1; in the synthesis process of compounds 2a, 2b, 2c, 2d, 2e, and 2f, in step 5, 3-cyano-4-hydroxy-benzoic acid methyl ester was used, 3-chloro-4-hydroxy-benzoic acid methyl ester, 3-nitro-4 hydroxy-methyl benzoate, 2-cyano-4 hydroxy-methyl benzoate, 2-chloro-4 hydroxy-methyl benzoate, and 2-nitro-4 hydroxy-methyl benzoate are used to replace 4-hydroxy-benzoic acid methyl ester, respectively, and the detailed preparation process will not be repeated here.

### Embodiment 2: Synthesis of Compounds 3a-4f

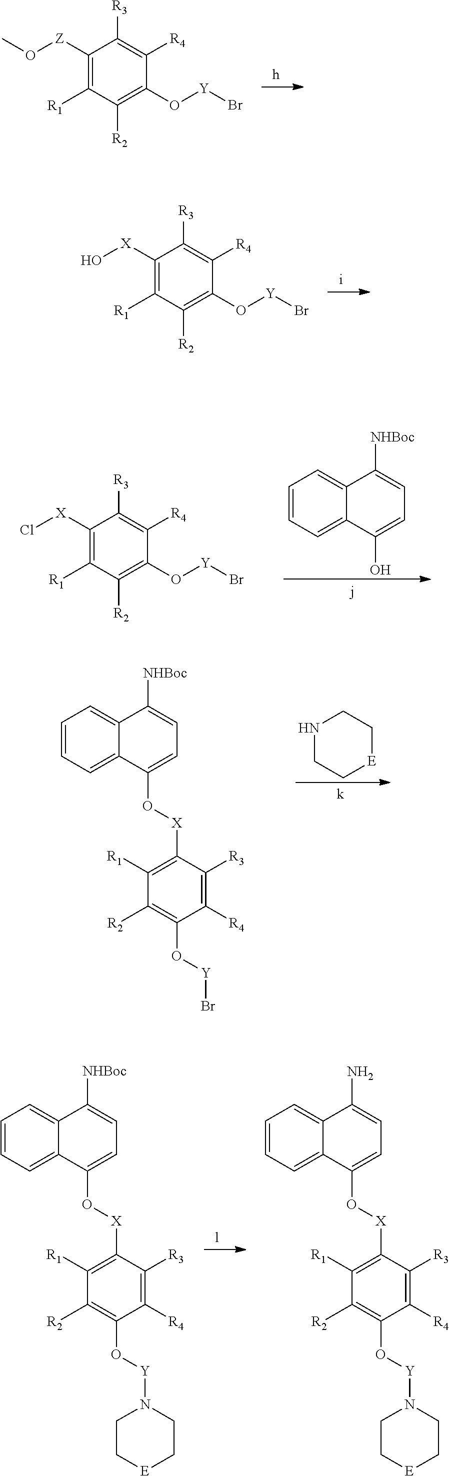 X=—CH₂—, Y=—(CH₂)₂—, Q=—CH₂—, E=—CH₂—, R₁=H, R₂=H, R₃=H, R₄=H; 3a:

X=—CH₂—, Y=—(CH₂)₃—, Q=—CH₂—, E=—O—, R₁=H, R₂=H, R₃=H, R₄=H; 3b:

X=—CH₂—, Y=—(CH₂)₂—, Q=—CH₂—, E=—(CH₂)₂—, R₁=H, R₂=H, R₃=H, R₄=H; 3c:

X=—CH₂—, Y=—(CH₂)₃—, Q=—CH₂—, E=—(CH₂)₂—, R₁=H, R₂=H, R₃=H, R₄=H; 3d:

X=—CH₂—, Y=—(CH₂)₃—, Q=—CH₂—, E=—CH₂—, R₁=H, R₂=H, R₃=H, R₄=H; 3e:

X=—CH₂—, Y=—(CH₂)₃—, Q=—CH₂—, E=—O—, R₁=H, R₂=H, R₃=H, R₄=H; 3f:

X=—CH₂—, Y=—(CH₂)₂—, Q=—CH₂—, E=—CH₂—, R₁=H, R₂=CN, R₃=H, R₄=H; 4a:

X=—CH₂—, Y=—(CH₂)₂—, Q=—CH₂—, E=—O—, R₁=H, R₂=Cl, R₃=H, R₄=H; 4b:

X=—CH₂—, Y=—(CH₂)₃—, Q=—CH₂—, E=—(CH₂)₂—, R₁=H, R₂=NO₂, R₃=H, R₄=H; 4c:

X=—CH₂—, Y=—(CH₂)₃—, Q=—CH₂—, E=—(CH₂)₂—, R₁=CN, R₂=H, R₃=H, R₄=H; 4d:

X=—CH₂—, Y=—(CH₂)₃—, Q=—CH₂—, E=—CH₂—, R₁=Cl, R₂=H, R₃=H, R₄=H; 4e:

X=—CH₂—, Y=—(CH₂)₃—, Q=—CH₂—, E=—O—, R₁=NO₂, R₂=H, R₃=H, R₄=H. 4f:

The specific synthesis method is as follows, taking the compound 3a as an example, whose structural formula is below:

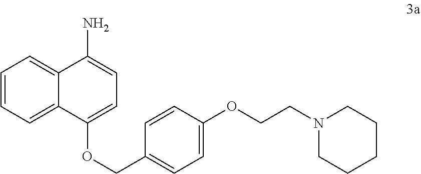 [SMILES: Nc1ccc(OCc2ccc(OCCN3CCCCC3)cc2)c2ccccc12]

Compound 3a is named 4-(4-(2-(piperidin-1-yl) ethoxy) benzyloxy)-1-naphthylamine dihydrochloride, and the synthetic route thereof is shown below:

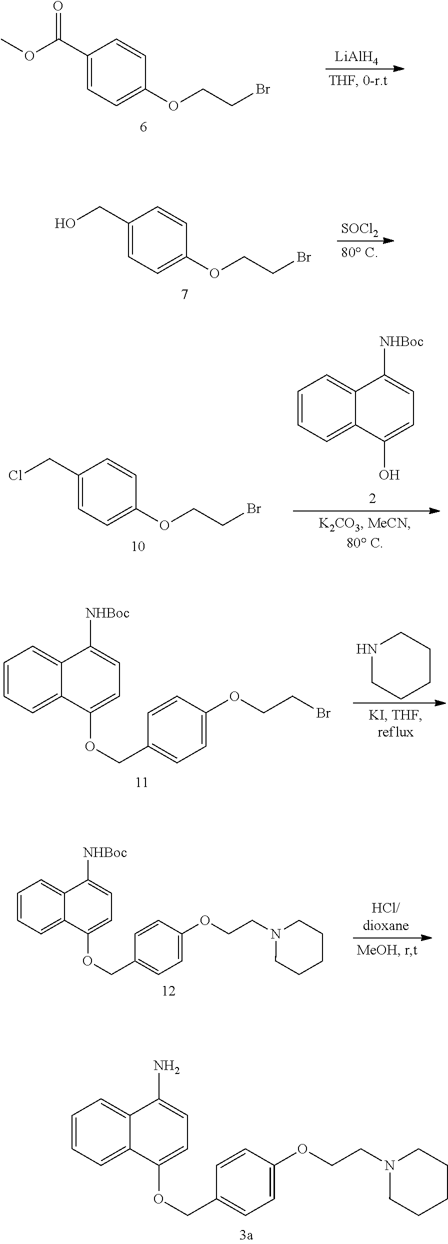

### Step 1. 4-(2-Bromoethoxy) benzyl Alcohol (9)

Dissolving methyl 4-(2-bromoethoxy) benzoate (6) (450 mg, 1.74 mmol, 1.0 eq) in 20 mL of anhydrous tetrahydrofuran, cooling to 0° C., adding lithium aluminum tetrahydrogen in batches, naturally heating up to room temperature to react for 0.5 hours. TLC (petroleum ether:ethyl acetate=3:1, R_{f}/compound 6=0.75, R_{f}/compound 9=0.30) shows that the reaction of the raw materials is completed and new spots are generated. Slowly pouring the reaction solution into 50 mL of water, adjusting the pH value to 1 with dilute hydrochloric acid (15%), extracting with 150 mL of ethyl acetate 3 times (50 mL*3), and combining the organic phases; drying with anhydrous sodium sulfate, filtering a layer of silica gel, spin-drying the filtrate to obtain 340 mg of 4-(2-bromoethoxy) benzyl alcohol (9), a colorless oily liquid, with a yield of 84.8%.

### Step 2. 4-(2-Bromoethoxy) benzyl Chloride (10)

Dissolving 4-(2-Bromoethoxy) benzyl alcohol (9) (340 mg, 1.47 mmol, 1.0 eq) in thionyl chloride (10 mL), and heating up to 80° C. to react for 2 hours. Spin-drying the reaction solution, dissolving in 20 mL of dichloromethane, and spin-drying again; repeating three times to obtain 365 mg of 4-(2-bromoethoxy) benzyl chloride (10), which is directly used in the next reaction without purification.

### Step 3. 4-(4-(2-Bromoethoxy) benzyloxy)-1-(tert-butoxycarbonyl) aminonaphthalene (11)

Dissolving 1-tert-butoxycarbonylamino-4-hydroxy-naphthalene (2) (380 mg, 1.47 mmol, 1.0 eq), 4-(2-bromoethoxy) benzyl chloride (10) (366 mg, 1.47 mmol, 1.0 eq) and potassium carbonate (405 mg, 2.93 mmol, 2.0 eq) in 15 mL of acetonitrile, and heating up to 80° C. for 12 hours. TLC (petroleum ether:ethyl acetate=3:1, R_{f}/compound 2=0.45, R_{f}/compound 11=0.75) shows that the reaction of the raw materials is completed and new spots are generated. Pouring the reaction solution into 50 mL of water, and extracting 3 times with 150 mL of ethyl acetate (50 mL*3); combining the organic phases, dryinh over anhydrous sodium sulfate, and spin-drying to obtain a crude product. Then purifying by column chromatography (petroleum ether:ethyl acetate=20:1-5:1) to obtain 250 mg of 4-(4-(2-bromoethoxy) benzyloxy)-1-(tert-butoxycarbonyl) aminonaphthalene (11), a yellow oily liquid, with a yield of 36.1%.

¹H NMR (CDCl₃, 300 MHz) δ: 7.84-7.77 (m, 2H), 7.48-7.39 (m, 2H), 7.28 (d, J=9.0 Hz, 2H), 7.07 (d, J=9.0 Hz, 2H), 6.83 (d, J=6.0 Hz, 2H), 6.54 (m, 1H), 4.35 (s, 2H), 4.22 (t, J=6.0 Hz, 2H), 3.56 (t, J=6.0 Hz, 2H), 1.51 (s, 9H).

### Step 4. 4-(4-(2-(piperidin-1-yl) ethoxy) benzyloxy)-1-(tert-butoxycarbonyl) aminonaphthalene (12)

Dissolving 4-(4-(2-Bromoethoxy) benzyloxy)-1-(tert-Butoxycarbonyl) aminonaphthalene (11) (250 mg, 0.53 mmol, 1.0 eq), piperidine (135 mg, 1.59 mmol, 3.0 eq) and potassium iodide (8.89 mg, 0.053 mmol, 0.10 eq) in 10 mL of tetrahydrofuran, and heating up to 78° C. to react for 12 hours. TLC (dichloromethane:methanol=10:1, R_{f}/compound 11=0.95, R_{f}/compound 12=0.30) shows that the reaction of the raw materials is completed and new spots are generated. Cooling the reaction solution to room temperature, pouring into 20 mL of water, and extracting three times with 60 mL of ethyl acetate reaction solution (20 mL*3); combining the organic phases and drying over anhydrous sodium sulfate and spin-drying. Purifying the crude product by column chromatography (gradient elution, dichloromethane:methanol=100:1-20:1) to obtain 60 mg of 4-(4-(2-(piperidin-1-yl) ethoxy) benzyloxy)-1-(tert-Butoxycarbonyl) aminonaphthalene (12), a pale yellow oily liquid, with a yield of 24.6%.

¹H NMR (CDCl₃, 300 MHz) δ: 7.82 (d, J=9.0 Hz, 1H), 7.76 (d, J=9.0 Hz, 1H), 7.44 (m, 2H), 7.27 (d, J=9.0 Hz, 2H), 7.08 (d, J=6.0 Hz, 1H), 6.83 (d, J=9.0 Hz, 2H), 6.48 (d, J=9.0 Hz, 1H), 4.49 (s, 2H), 4.14 (t, J=6.0 Hz, 2H), 2.84 (t, J=6.0 Hz, 2H), 2.60 (m, 4H), 1.65 (m, 4H), 1.51 (s, 9H), 1.43 (m, 2H).

### Step 5. 4-(4-(2-(piperidin-1-yl) ethoxy) benzyloxy)-1-naphthylamine Dihydrochloride (Hydrochloride of Compound 3a)

Dissolving 4-(4-(2-(piperidin-1-yl) ethoxy) benzyloxy)-1-(tert-butoxycarbonyl) aminonaphthalene (12) in 2 mL of methanol, slowly adding 2 mL HCl/methanol solution (6 mol/L) with stirring, and reacting at room temperature for 12 hours. TLC (dichloromethane:methanol=10:1, R_{f}/compound 12=0.30, R_{f}/compound 2a=0.15) shows that the reaction of the raw materials is completed and new spots are generated. Spin-drying the reaction solution and stripping with anhydrous toluene three times to obtain 45 mg of 4-(4-(2-(piperidin-1-yl) ethoxy) benzyloxy)-1-naphthylamine dihydrochloride (2a), a brown oily liquid, with a yield of 81.8%. ¹H NMR (DMSO-d⁶, 300 MHz) δ: 10.5 (brs, 2H), 8.20 (d, J=9.0 Hz, 1H), 8.13 (d, J=9.0 Hz, 2H), 7.63-7.52 (m, 2H), 7.34-7.21 (m, 3H), 6.92 (d, J=9.0 Hz, 1H), 6.82 (d, J=9.0 Hz, 1H), 4.50 (s, 2H), 4.37 (t, J=6.0 Hz, 2H), 3.55-3.30 (m, 4H), 2.97 (t, J=6.0 Hz, 2H), 1.79-1.67 (m, 4H), 1.65 (m, 4H), 1.39-1.20 (m, 2H).

Refer to Embodiment 2 for the synthesis methods of compounds 3b to 4f, the difference is: in the synthesis process of compounds 3b and 3c, in step 4, morpholine and homopiperidine are used to replace piperidine, respectively, and the remaining steps are the same as in Embodiment 2; in the synthesis process of compounds 3d, 3e, and 3f, in step 5 of Embodiment 2, 1, 3-dibromopropane is used to replace 1, 2-dibromopropane; in the synthesis process of compound 3d and 3f, in step 4, morpholine and homopiperidine are used to replace piperidine, respectively, and the remaining steps are the same as in Embodiment 2; in the synthesis process of compounds 4a, 4b, 4c, 4d, 4e, and 4f, in step 5 of Embodiment 2, 3-cyano-4-hydroxy-benzoic acid methyl ester, 3-chloro-4 hydroxy-benzoic acid methyl ester, 3-nitro-4 hydroxy-benzoic acid methyl ester, 2-cyano-4-hydroxy-methyl benzoate, 2-chloro-4 hydroxy-methyl benzoate, and 2-nitro-4 hydroxy-methyl benzoate are used to replace 4-hydroxy-benzoic acid methyl ester, and the detailed preparation process will not be repeated here.

Experiment on Inducing Apoptosis of Breast Cancer, Colon Cancer and Lung Cancer Cancer Cells

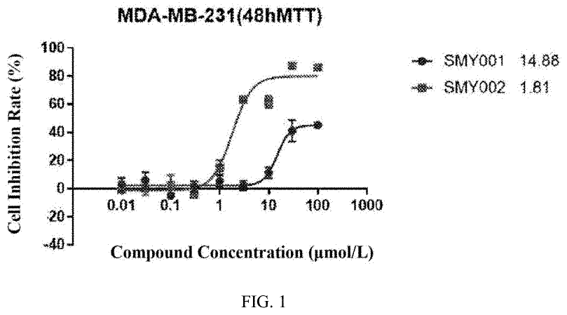

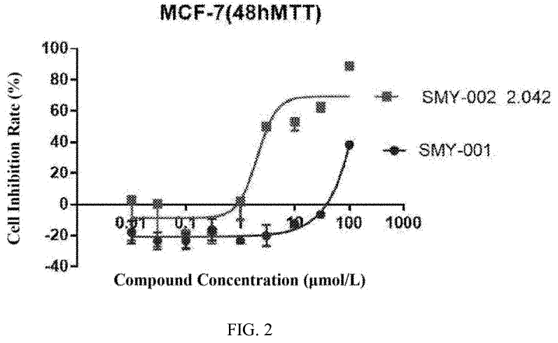

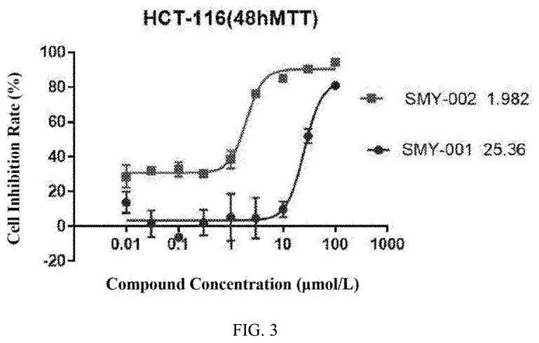

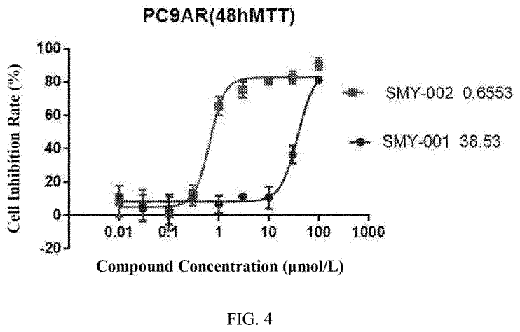

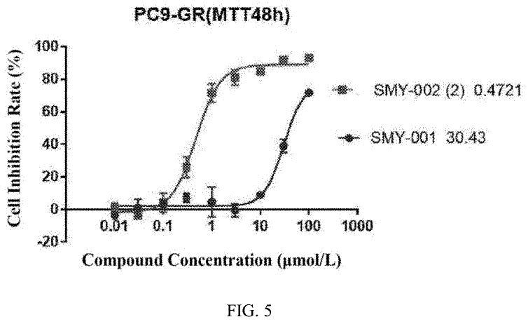

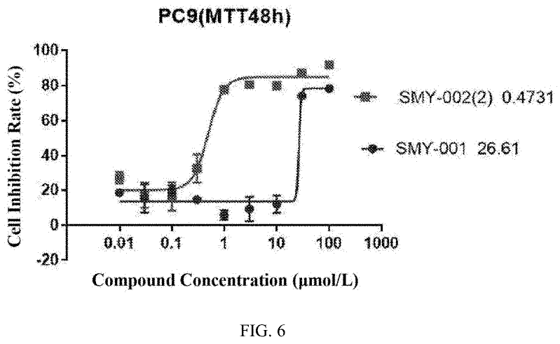

Method: collecting logarithmic growth phase MDA-MB-231, MCF-7, HCT-116, PC9-AR, PC9-GR, PC9 cells, counting, adjusting the cell suspension concentration to 50000 cells/mL, adding 100 ul cell suspension to each well, that is, 5000 cells per well, and adding the above cancer cells to the hydrochloride of the naphthylamine compounds of 1a and 3a of the invention, to enable the final concentration of the hydrochloride of the naphthylamine compound in the system to be several gradients of 0.1, 0.3, 1, 3, 10, 30, 100, 300 (μmol/L), and continuing culturing for 48 hours; after treating with drug, adding 50 μL of Thiazole Blue reagent (1 mg/mL) to each well, incubating at 37° C. for 4 hours, shaking the plate to discard the liquid in the well, draining the water, absorbing the remaining liquid with filter paper, and then adding 100 μL of dimethyl sulfoxide; reacting on a horizontal shaker for 7-8 minutes, until the blue-violet crystals are completely dissolved; reading the value with a microplate reader, measuring the OD value at the absorption wavelength of 570 nm, recording the result. The graphs of the added concentration and cell inhibition rate of 1a (corresponding to SMY-001 in FIG. 1 to 6) and 3a (corresponding to SMY-002 in FIG. 1 to 6) are shown in FIG. 1 to 6, and the statistical results of the hydrochlorides of the naphthylamine compounds of 1a and 3a are shown in the following table:

| | | Chemically | Molecular | | IC50 |
|---|---|---|---|---|---|
| Name | Molecular Structure | Nomenclature | Weight | Cell Strain | (μmol/L) |
| SMY001 (1a) | 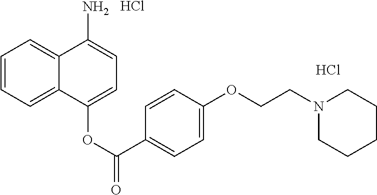 [SMILES: Cl ; Nc1ccc(OC(=O)c2ccc(OCCN3CCCCC3)cc2)c2ccccc12 ; Cl] | 4-aminonaphthalen-1- yl 4-(2-(piperidin-1-yl) ethoxy) benzoate dihydrochloride | 463.4 | MCF-7 MDA-MB-231 HCT-116 PC9AR PC9GR PC9 | Null 14.88 25.36 38.53 30.43 26.61 |
| SMY002 (3a) | 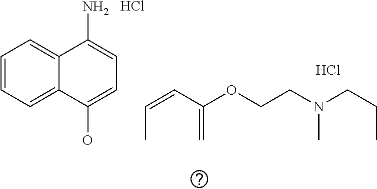 | 4-((4-(2-( piperidin-1- yl) ethoxy) benzyl) oxy) naphthalene-1-amine dihydrochloride | 449.42 | MCF-7 MDA-MB-231 HCT-116 PC9AR PC9GR PC9 | 2.042 1.81 1.982 0.6553 0.4721 0.4731 |

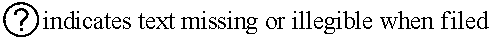

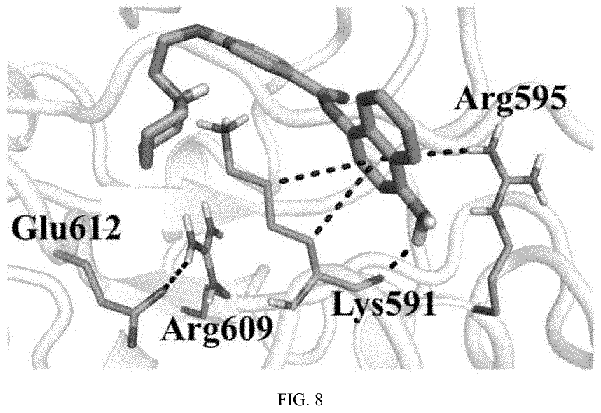

The table shows that SMY001 and SMY002 have significant inhibitory effects on breast cancer cell lines (MCF-7, MDA-MB-231), human colon cancer (HCT-116) and lung cancer cell lines (PC9, PC9AR and PC9GR) at low concentrations. According to the conclusion of molecular simulation (FIG. 8), SMY001 has a significant interaction with the phosphorylated tyrosine kinase region of STAT3-SH2 functional domain. The naphthylamine group of SMY001 (as a common group of the naphthylamine compound of formula I) participates in the polar and hydrophobic interactions with the key amino acids-lysine 591 and arginine 595, respectively. Therefore, the naphthylamine group of SMY001 is not only a common group for the naphthylamine compound of formula I, but also a key group involved in the interaction with protein molecules. It is inferred that the amino group of the naphthylamine compound of formula I has a strong interaction with the phosphorylated tyrosine interaction region of the SH2 functional domain of the STAT3 protein; the naphthylamine compounds of formula I are all inhibitors of STAT3 acting on lysine 591, arginine 595 and arginine 609. Therefore, the naphthylamine compound of formula I can inhibit the binding of STAT3 protein to upstream and downstream proteins in signal transduction, inhibit the phosphorylation of STAT3 protein, block the expression of downstream genes in STAT3 signal transduction, induce apoptosis of related tumor cells, and achieve the effect of controlling tumor growth.

Westernblot Experiment

1. Cell Culturing and Dosing

(1) Taking HCC827 cells in logarithmic growth phase, digesting them with trypsin, preparing a single cell suspension with a density of 300,000 cells/mL with RPMI-1640 medium containing 10% fetal bovine serum, and adding 2 mL of cell suspension to each well to inoculate a 6-well cell culture plate.

(2) Incubating in a 37° C., 5% CO₂ incubator. After the cells adhere to the wall, adding the experimental group with different concentrations of the drug SMY002 (3a), and the concentration gradients are: 10, 30, 100 and 300 μmol/L; adding 30 μL of interleukin-6 (IL-6) one hour later at a concentration of 1 mg/mL to stimulate the cells, and the final concentration of interleukin-6 (IL-6) is 30 ng/mL.

(3) After continuing to culture for 0.5 h, lysing the cells with RIPA lysis buffer to collect proteins.

2. Cell Collection and Lysis

(1) Removing the upper layer of culture medium, and washing the cells in the 6-well plate twice with phosphate buffered saline (PBS). Adding 160 μL of pre-chilled RIPA cell lysate (protease inhibitor and phenylmethylsulfonyl fluoride and lysate are added in advance at a ratio of 1:100). Scraping the cell lysate with the cell scraper that is washed in advance, and collecting it into a clean 1.5 mL centrifuge tube.

(2) Placing on ice, lysing for 30 minutes, vortexing once every certain time (6 minutes).

(3) Centrifuging for 12 minutes at 4° C., 12000 rpm.

(4) Transferring the cell supernatant to a clean centrifuge tube. Dividing the cell supernatant into two parts: taking 5 μL and add it to a 1.5 mL centrifuge tube for BCA to measure the protein content, then adding 45 μL of 1× phosphate buffered saline (PBS) and mixing well for later use; quantitatively taking 140 μL of the remaining cell supernatant, adding 35 μL of 5×SDS loading buffer, boiling in boiling water for 8 minutes after mixing, and storing in a refrigerator at −20° C. after centrifugation.

(5) Procedures for protein concentration determination:

A. 1× Phosphate Buffer Saline (PBS) diluted protein standard:

| Tube | | | BSA Standard |
|---|---|---|---|
| Number | 1 × PBS (μL) | BSA Standard Dosage | (μg/mL) |
| A | 0 | 100 | 2000 |
| B | 200 | 200 | 1000 |
| C | 200 | 200 (taken from Tube B) | 500 |
| D | 200 | 200 (taken from Tube C) | 250 |
| E | 200 | 200 (taken from Tube D) | 125 |
| F | 400 | 100 (taken from Tube E) | 25 |
| G | 200 | 0 | 0 (null) |

B. Preparation of BCA working fluid: according to the number of standards and samples to be tested, calculating the total amount of A and B mixed working fluid required. According to the ratio of BCA reagent A to B volume ratio of 50:1, preparing the working solution, vortexing and shaking to mix well for later use.

C. Adding 25 μL each of the protein standard solution and the sample supernatant diluted with phosphate buffered saline (PBS) (10-fold dilution) to a new 96-well plate. Then adding 200 μL of pre-prepared BCA working solution and mixing well. Remember not to generate bubbles by pipetting, closing the 96-well plate cover tightly, and reacting for 30 minutes in a 37° C. incubator.

D. Taking out the 96-well plate and return to room temperature for 3-5 minutes, measuring the absorbance value at 562 nm wavelength on the microplate reader, and creating a standard curve to calculate the content of 1 μL/Protein of each sample for protein loading.

3. Sodium Dodecyl Sulfonate-Polyacrylamide Gel (SDS-PAGE)

(1) Fixing the glue plate and preparing 10% SDS-PAGE separating glue.

Preparing 10 mL of the separation gel according to the following table:

| | |
|---|---|
| Deionized Water | 4.0 mL |
| 30% (m/v) Acrylamide | 3.3 mL |
| 1.5 M Tris-HCI (PH 8.8) Buffer | 2.5 mL |
| 10% (m/v) Sodium Dodecyl Sulfonate | 0.1 mL |
| 10% (m/v) Ammonium Persulfate | 0.1 mL |
| Tetramethylethylenediamine | 4 μL |
| Total | 10 mL |

(2) Adding the mixed separation glue to two rubber plates respectively, adding them to a position 1.0 cm from the top, filling the rubber plates with absolute ethanol, and keeping them stand for 30-45 minutes.

(3) After the separation glue is congealed, pouring out the remaining absolute ethanol, and using filter paper to absorb the remaining absolute ethanol.

(4) Preparing 5 mL of 5% concentrated glue according to the following table.

| | |
|---|---|
| Deionized Water | 2.77 mL |
| 30% (m/v) Acrylamide | 830 μL |
| 0.5 M Tris-HCI (PH 6.8) Buffer | 1.26 mL |
| 10% (m/v) Sodium Dodecyl Sulfonate | 50 μL |
| 10% (m/v) Ammonium Persulfate | 50 μL |
| Tetramethylethylenediamine | 5 μL |
| Total | 5 mL |

(5) Slowly adding the prepared concentrated glue to the rubber plate to avoid air bubbles, inserting the sample comb, and keeping it stand for 30-45 minutes.

(6) Taking out the protein sample, heating it in a water bath at 100° C. for 5 minutes, rotating at 10000 rpm, and centrifuging for 10 minutes.

(7) Fixing the gel plate in the electrophoresis tank, adding SDS-PAGE electrophoresis buffer, pulling out the sample comb, and adding the processed protein samples to the sample tank in order.

(8) Electrophoresis at 80 V for 40 minutes.

(9) Changing the voltage to 120V for electrophoresis for about 1.5 hours until the bromophenol blue runs out of the colloid;

4. Western-Blot Membrane Transfer

(1) Putting the SDS-PAGE gel after electrophoresis in the TBST buffer and rinsing once, and putting the protein gel in the membrane transfer buffer to soak.

(2) Soaking a layer of sponge pad in the membrane transfer buffer and clamping it on the membrane transfer instrument with a tweezer; putting the sponge pad, three layers of filter paper, protein glue, polyvinylidene fluoride (PVDF) membrane, three layers of filter paper, and sponge pad in order, aligning them, and putting them on the membrane transfer instrument; during the operation, the filter paper and sponge pad should be soaked in the membrane transfer buffer. If there are bubbles between each layer, using a glass test tube to gently roll them out.

(3) Turning on the membrane transfer instrument and performing membrane transfer at 300 mA for 75 minutes.

(4) Taking the membrane out, putting it in TBST buffer, rinsing 3 times with a 60 rpm horizontal shaker, 8 minutes each time.

(5) Using 20 mL of 5% bovine serum albumin (BSA) blocking solution, 60 rpm horizontal shaker to block at room temperature for 2 hours.

(6) Using 3 mL of antibody incubation solution with 3 μL of primary antibodies (Stat3 and p-STAT3 1:1000), and incubating overnight at 4° C. and 60 rpm in a horizontal shaker.

(7) Washing the PVDF membrane three times with a 10 mL TBST, 60 rpm horizontal shaker at room temperature, 10 minutes each time.

(8) Incubating the PVDF membrane with a 20 mL antibody incubation solution containing 2 μL of secondary antibody and a horizontal shaker at room temperature at 60 rpm for 2 hours.

(9) Washing the PVDF membrane three times with a 10 mL TBST, 60 rpm horizontal shaker at room temperature, 10 minutes each time.

(10) Taking 1 mL each of chemiluminescence substrate reagent solution A and solution B, and developing color at room temperature for 5 minutes.

(11) Using filter paper to absorb the liquid on the membrane and developing it with a developing device.

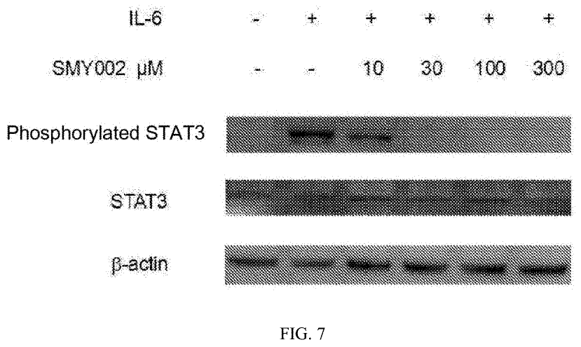

FIG. 7 is the result of Westernblot of the compound SMY002 (3a). The result of Westernblot experiment is to transfer the total cell protein after electrophoresis separation from the gel to the solid support membrane. According to the specific principle of antigen and antibody, the corresponding protein expression is detected by STAT3, p-STAT3, and β-Actin antibodies respectively. The results are shown in the figure; it can be seen that under the action of the drug, with the increase of drug concentration, the amount of STAT3 and β-Actin protein expressed by HCC827 remains unchanged, while the expression amount of p-STAT3 shows a downward trend. The compound SMY002 (3a) significantly inhibits the expression of p-STAT3.

Molecular Docking Experiment

Method: in order to verify the interaction mechanism between the compound SMY001 and STAT3 protein, the inventor uses the phosphorylated tyrosine (pY-705) binding region of the SH2 domain of the STAT3 protein as a protein template for computer virtual simulation (docking), and the virtual docking region is mainly concentrated in the area near the phosphorylated tyrosine sites ARG609 and LYS591. The structure coordinates of STAT3 SH2 are taken from the protein structure database (PDB data bank, ID:1BG1). Molecular docking method: all computer docking experiments are completed on the sybyl X2.1.1 operating platform, and the computer docking tool used is SUEFLEX DOCK. Performing calculations based on the selected sites (mainly including phosphorylated tyrosine sites ARG609 and LYS591) to determine the potential gradient and conduct computer docking experiments. Analysis is based on docking scores and conformations and interactions. The virtual docking in FIG. 8 shows that SMY001 (1a) interacts with the lysine 591 and arginine 595 of the STAT3-SH2 functional domain with Pi-Pi folding, and has a polar interaction with the main chain of lysine 591.

According to the conclusion of molecular simulation (FIG. 8), the amino group of the naphthylamine compounds of formula I have a strong interaction with the phosphorylated tyrosine interaction region of the SH2 domain of the STAT3 protein; the naphthylamine compounds of formula I are all inhibitors of STAT3 acting on lysine 591, arginine 595 and arginine 609; therefore, the naphthylamine compounds of formula I can inhibit the binding of STAT3 protein to upstream and downstream proteins in signal transduction, inhibit the phosphorylation of STAT3 protein, block the expression of downstream genes in STAT3 signal transduction, induce apoptosis of related tumor cells, and achieve the role of controlling tumor growth.

Therefore, according to the general route (first carry out routine anti-tumor in vitro screening, and then carry out targeted research) of drug development, the compound of the invention can be applied to cancer treatment drugs related to abnormal signal transduction of STAT3 cells, and anti-tumor drugs can be prepared by forming salts with human-acceptable acids or mixing with pharmaceutical carriers.

Finally, it should be noted that the above embodiments are only used to illustrate rather than limit the technical solutions of the invention. Any equivalent substitutions made to the invention and modifications or partial substitutions without departing from the spirit and scope of the invention shall all fall within the protection scope of the invention.

1. A naphthylamine compound, wherein the structural formula thereof is as shown in general formula I: 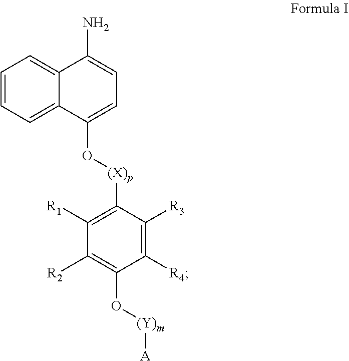
R₁, R₂, R₃, and R₄ are each independently selected from hydrogen, halogen, nitro, alkyl, cyano, and aryl;
p represents the number of X substituents, and P is 0 or 1;
X is —CH₂—, —(CH₂)₂—, —CO—, —CH₂—CO— or —(CH₂)₂—CO—;
m represents the number of Y substituents, and M is 0 or 1;
Y is —(CH₂)₂—, —(CH₂)₃—, —CO—, —CH₂—CO— or —(CH₂)₂—CO—;
A is 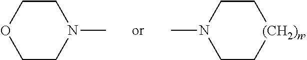
wherein n=0, 1, 2, 3.

2. The naphthylamine compound according to claim 1, wherein it is specifically a compound with the following structure:

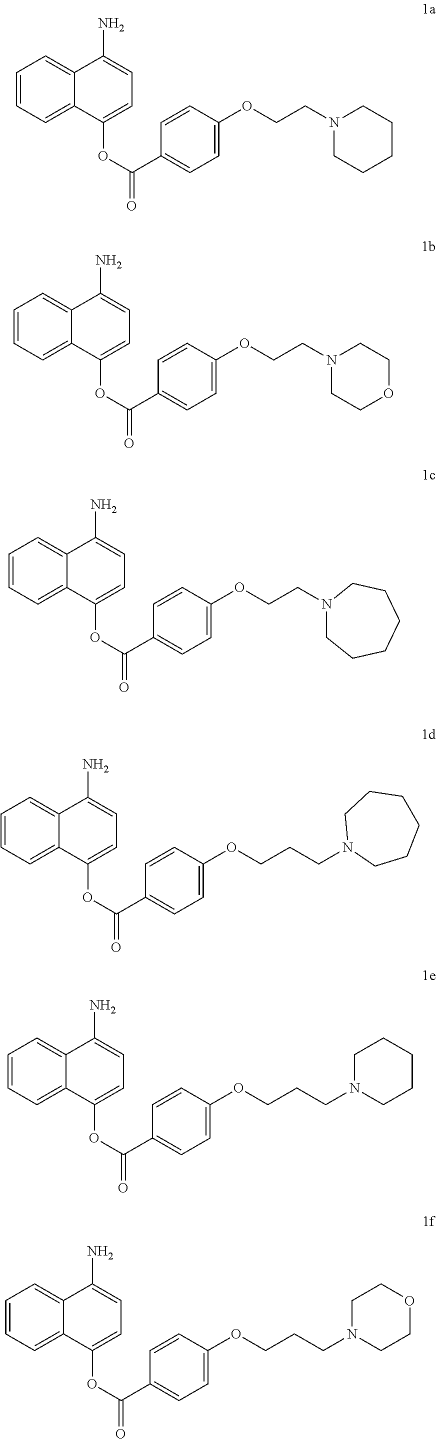 [SMILES: Nc1ccc(OC(=O)c2ccc(OCCN3CCCCC3)cc2)c2ccccc12 ; Nc1ccc(OC(=O)c2ccc(OCCN3CCOCC3)cc2)c2ccccc12 ; Nc1ccc(OC(=O)c2ccc(OCCN3CCCCCC3)cc2)c2ccccc12 ; Nc1ccc(OC(=O)c2ccc(OCCCN3CCCCCC3)cc2)c2ccccc12 ; Nc1ccc(OC(=O)c2ccc(OCCCN3CCCCC3)cc2)c2ccccc12 ; Nc1ccc(OC(=O)c2ccc(OCCCN3CCOCC3)cc2)c2ccccc12]

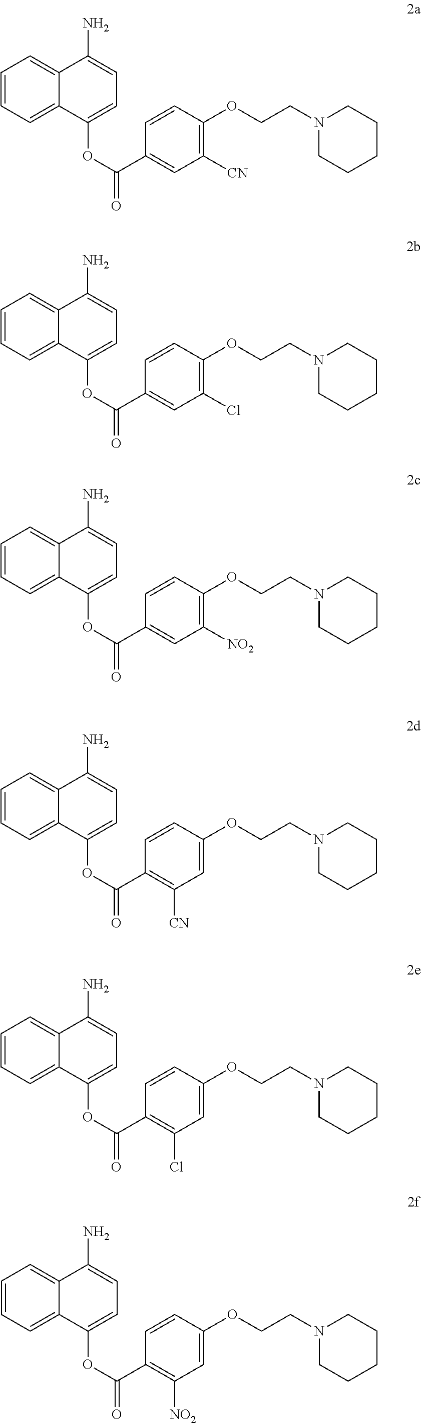 [SMILES: N#Cc1cc(C(=O)Oc2ccc(N)c3ccccc23)ccc1OCCN1CCCCC1 ; Nc1ccc(OC(=O)c2ccc(OCCN3CCCCC3)c(Cl)c2)c2ccccc12 ; Nc1ccc(OC(=O)c2ccc(OCCN3CCCCC3)c([N+](=O)[O-])c2)c2ccccc12 ; N#Cc1cc(OCCN2CCCCC2)ccc1C(=O)Oc1ccc(N)c2ccccc12 ; Nc1ccc(OC(=O)c2ccc(OCCN3CCCCC3)cc2Cl)c2ccccc12 ; Nc1ccc(OC(=O)c2ccc(OCCN3CCCCC3)cc2[N+](=O)[O-])c2ccccc12]

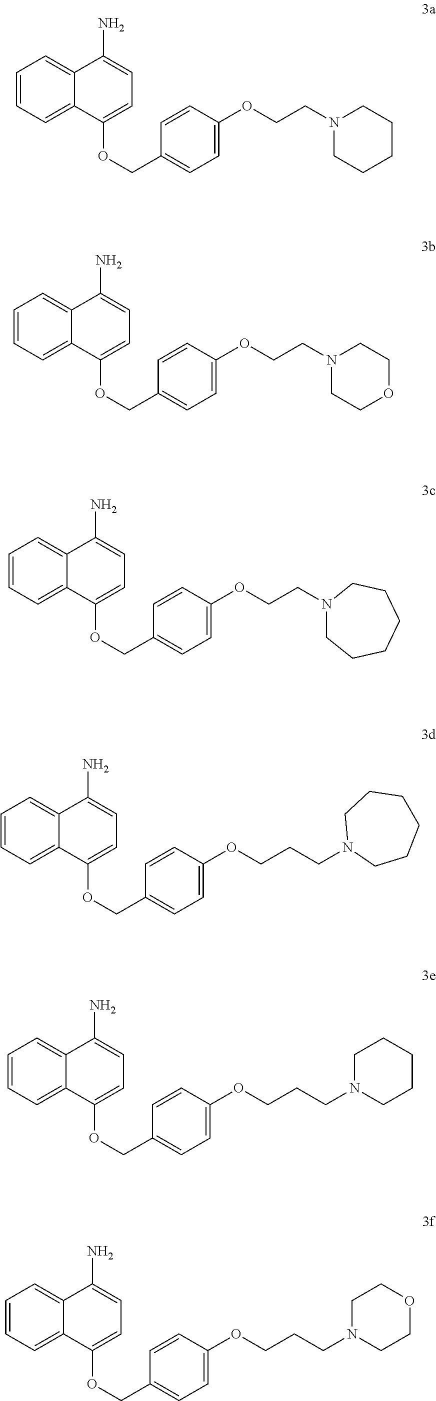 [SMILES: Nc1ccc(OCc2ccc(OCCN3CCCCC3)cc2)c2ccccc12 ; Nc1ccc(OCc2ccc(OCCN3CCOCC3)cc2)c2ccccc12 ; Nc1ccc(OCc2ccc(OCCN3CCCCCC3)cc2)c2ccccc12 ; Nc1ccc(OCc2ccc(OCCCN3CCCCCC3)cc2)c2ccccc12 ; Nc1ccc(OCc2ccc(OCCCN3CCCCC3)cc2)c2ccccc12 ; Nc1ccc(OCc2ccc(OCCCN3CCOCC3)cc2)c2ccccc12]

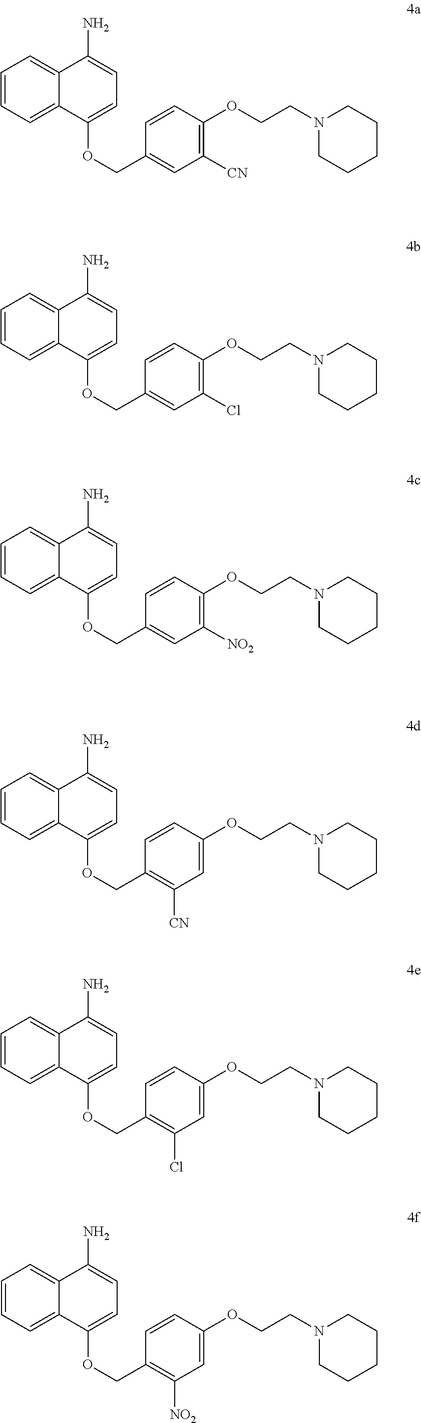 [SMILES: N#Cc1cc(COc2ccc(N)c3ccccc23)ccc1OCCN1CCCCC1 ; Nc1ccc(OCc2ccc(OCCN3CCCCC3)c(Cl)c2)c2ccccc12 ; Nc1ccc(OCc2ccc(OCCN3CCCCC3)c([N+](=O)[O-])c2)c2ccccc12 ; N#Cc1cc(OCCN2CCCCC2)ccc1COc1ccc(N)c2ccccc12 ; Nc1ccc(OCc2ccc(OCCN3CCCCC3)cc2Cl)c2ccccc12 ; Nc1ccc(OCc2ccc(OCCN3CCCCC3)cc2[N+](=O)[O-])c2ccccc12]

3. A biologically acceptable salt formed by the naphthylamine compound with at least one of acetic acid, dihydroacetic acid, benzoic acid, citric acid, sorbic acid, propionic acid, oxalic acid, fumaric acid, maleic acid, hydrochloric acid, malic acid, phosphoric acid, sulfurous acid, sulfuric acid, vanillic acid, tartaric acid, ascorbic acid, boric acid, lactic acid and ethylenediaminetetraacetic acid.

4. A preparation method of the naphthylamine compound, comprising the following steps:
(1) dissolving 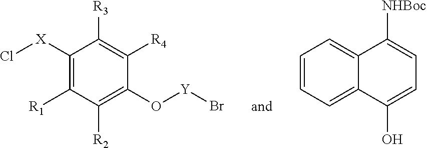
with a molar ratio of 1:1 in an organic solvent, and adding an alkali; after the reaction is detected by TLC, 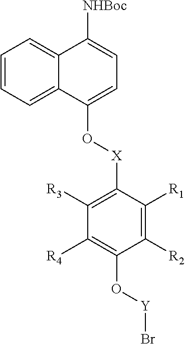
is obtained by post-processing;
(2) then 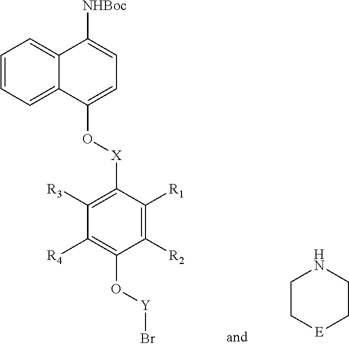
with a molar ratio of 1:3 are subjected to a nucleophilic substitution reaction to generate 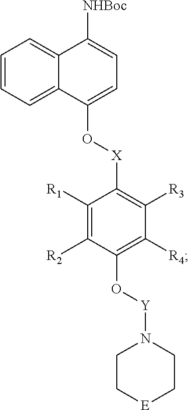
wherein E is —CH₂—, —O— or —(CH₂)₂—.

5. The preparation method of the naphthylamine compound according to claim 4, wherein the 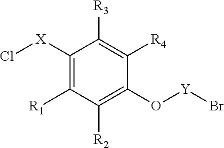
is prepared by the following method: 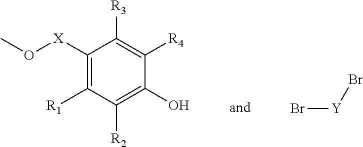
with a molar ratio of 1:4 are subjected to a nucleophilic substitution reaction to obtain 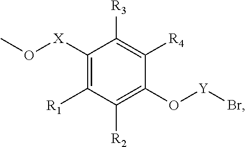
and then 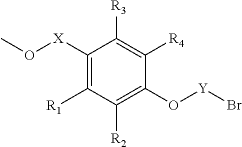
is converted to 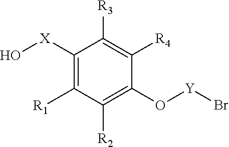
through the reaction, and then is subjected to a halogenating reaction with a chlorinating agent to obtain the

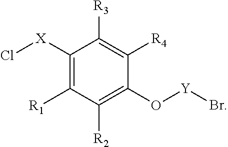

6. The preparation method of the naphthylamine compound according to claim 5, wherein the 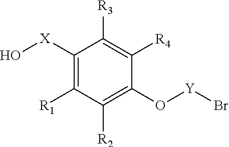
comprises 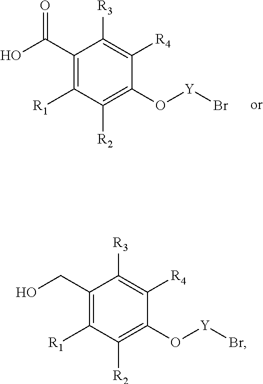
and the specific preparation method of 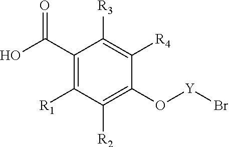
is as follows:
dissolving the 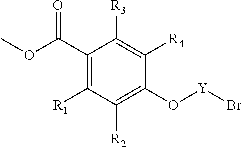
in a mixed solvent of tetrahydrofuran and water, then adding lithium hydroxide, and reacting at 20-50° C.; after the reaction is detected by TLC, removing the tetrahydrofuran by rotary evaporation; adjusting the pH value of the residue to 1-3 with hydrochloric acid, and the solid obtained by precipitation is 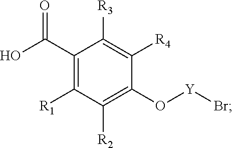
the specific preparation method of 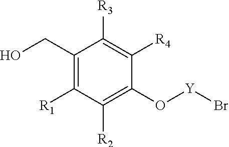
is as follows:
dissolving 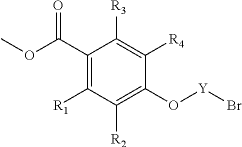
in tetrahydrofuran, adding lithium aluminum tetrahydrogen, and reacting at room temperature; after the reaction is detected by TLC, pouring the reaction solution into water; adjusting the pH value to 1-3 with hydrochloric acid, extracting with ethyl acetate, collecting the organic phase, filtering and performing rotary evaporation to obtain it.

7. The preparation method of the naphthylamine compound according to claim 6, wherein when the structural formula of the compound is 1a to 2f, Y is —(CH₂)₂— or —(CH₂)₃— at the moment, and the specific preparation method thereof is as follows:
(1) dissolving 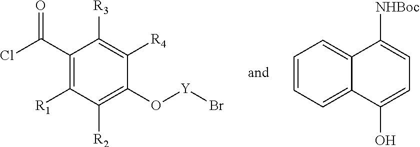
in tetrahydrofuran, adding triethylamine, and reacting at room temperature; after the reaction is detected by TLC, 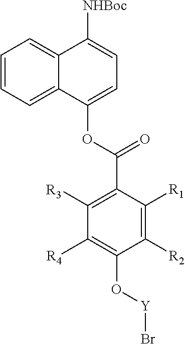
is obtained by post-processing;
wherein the molar ratio of 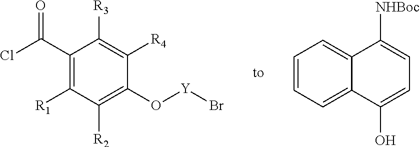
to triethylamine is 1:1:2;
(2) then dissolving 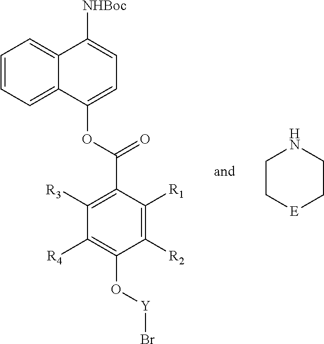
in tetrahydrofuran, adding potassium iodide, and after the reflux reaction is completed, it is obtained by post-processing;
wherein the molar ratio of 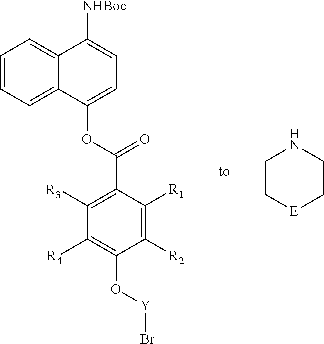
to potassium iodide is 1:3:0.1.

8. The preparation method of the naphthylamine compound according to claim 6, wherein when the structural formula of the compound is 3a to 4f, Y is —(CH₂)₂— or —(CH₂)₃— at the moment, and the specific preparation method thereof is as follows:
(1) dissolving 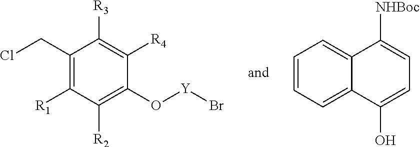
in acetonitrile, adding potassium carbonate, and reacting at 60-80° C.; after the reaction is detected by TLC, 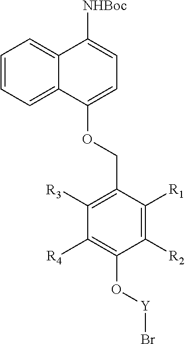
is obtained by post-processing;
wherein the molar ratio of 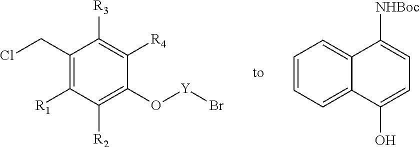
to potassium carbonate is 1:1:1.2;
(2) then dissolving 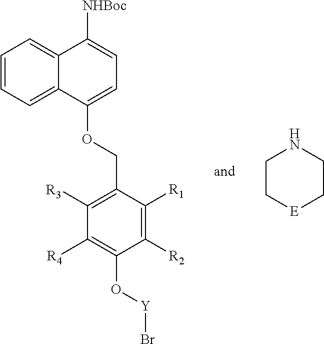
in tetrahydrofuran, adding potassium iodide, and after the reflux reaction is completed, it is obtained by post-processing;
wherein the molar ratio of 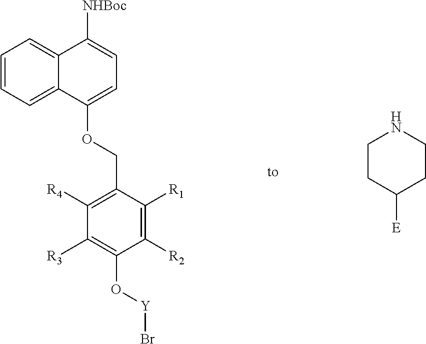
to potassium iodide is 1:3:0.1.

9. The biologically acceptable salt of the naphthylamine compound according to claim 3, wherein it is prepared by the following method: dissolving the naphthylamine compound in the methanol solution of the corresponding acid, and reacting at room temperature; after the reaction is detected by TLC, it is obtained by post-processing.

10. An application of the naphthylamine compound and the biologically acceptable salt thereof in the preparation of a medicament for the treatment of diseases related to STAT3 cell signal transduction.
